SEQUENCE LISTING (37 sequences)

SEQ ID NO: 1 - DNA, 53 bp, synthetic construct
  source 1..53: mol_type = other DNA
gttattactcgcggcccagccggccatggctgatatccagatgatccagagcc

SEQ ID NO: 2 - DNA, 60 bp, synthetic construct
  source 1..60: mol_type = other DNA
ccatgatggtggtgatggtgttggcctcccgggccactagtggcagacacggtaaccagg

SEQ ID NO: 3 - protein, 241 aa, synthetic construct
DIQMIQSQKFMSTSVGDRVTVTCKASQNVGTNVAWYQQKPGQSPNALIYSASYRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTNYFIYWVKQRPGQGLEWIGEINPRNGDTDFNEKFESRATLTVDKSSSTAYMQLSSLTSEDSAIYYCTRSPYGNNYGFTYWGQGTLVTVSA

SEQ ID NO: 4 - protein, 241 aa, synthetic construct
DIQMIQSQKFMSTSVGDKVTVTCKASQNVGTNVAWYQQKPGQSPNALIYSASYRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTKYFIYWVKQRPGQGLEWIGEINPRNGDTNFNEKFESRATLTVDKSSSTAYMQLSSLTSEDSAIYYCTRSPYGNNYGFTYWGQGTLVTVSA

SEQ ID NO: 5 - protein, 241 aa, synthetic construct
DIQMIQSQKFMSTSVGDRVTVTCKASQNVGTNVAWYQQKPGQSPNALIYNASHRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTNYFIYWVKQRPGQGLEWIGEINPRNGDTDFNEKFESRATLTVDKSSSTAYMQLSSLTSEDSAIYYCTRSPYGNNYGFTYWGQGTLVTVSA

SEQ ID NO: 6 - protein, 241 aa, synthetic construct
DIQMIQSQKFMSTSVGDRVTVTCKASQNVGTNVAWYQQKPGQSPNALIYSASYRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTNYFIHWVKQRPGQGLEWIGEINPRNGDTDFNEKFESRATLTVDKSSSTAYMQLSSLTSEDSAIYYCTRSPYGNNYGFTYWGQGTLVTVSA

SEQ ID NO: 7 - protein, 241 aa, synthetic construct
DIQMIQSQKFMSTSVGDRVTVTCKASQNVGTNVAWYQQKPGQSPNALIYSASYRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTNYFIHWVKQRPGQGLEWIGEINPKNGDTGFNEKFESRATLTVDKSSSTAYMQLSSLTSEDSAIYYCTRSPYGNNYGFTYWGPGTLVTVSA

SEQ ID NO: 8 - protein, 241 aa, synthetic construct
DIQMIQSQKFMSTSVGDRVTVTCKASQNVGTNVAWYQQKPGQSPNALIYSASYRYSGVPDRFTGSGSGTDFTLTITNVQSEDLADYFCQQYNSYPLTFGTGTKLDLKGGGGSGGGGSGGGGSVQLQQSGAELVKPGASVKLSCKASGYTFTNYFIYWVKQRPGQGLEWIGEINPRNGDTDFNVKFKSRATLTVDKSSSTAYMQLSSLTSEDSAIYYCTRSPYRNNYGFTYWGQGTLVTVSA

SEQ ID NO: 9 - protein, 107 aa, synthetic construct
DIQMIQSQKFMSTSVGDRVTVTCKASQNVGTNVAWYQQKPGQSPNALIYSASYRYSGVPDRFTGSGSGTDFTLTITNVQSEDLADYFCQQYNSYPLTFGTGTKLDLK

SEQ ID NO: 10 - protein, 119 aa, synthetic construct
VQLQQSGAELVKPGASVKLSCKASGYTFTNYFIYWVKQRPGQGLEWIGEINPRNGDTDFNEKFESRATLTVDKSSSTAYMQLSSLTSEDSAIYYCTRSPYGNNYGFTYWGQGTLVTVSA

SEQ ID NO: 11 - protein, 107 aa, synthetic construct
DIQMIQSQKFMSTSVGDKVTVTCKASQNVGTNVAWYQQKPGQSPNALIYSASYRYSGVPDRFTGSGSGTDFTLTITNVQSEDLADYFCQQYNSYPLTFGTGTKLDLK

SEQ ID NO: 12 - protein, 119 aa, synthetic construct
VQLQQSGAELVKPGASVKLSCKASGYTFTKYFIYWVKQRPGQGLEWIGEINPRNGDTNFNEKFESRATLTVDKSSSTAYMQLSSLTSEDSAIYYCTRSPYGNNYGFTYWGQGTLVTVSA

SEQ ID NO: 13 - protein, 107 aa, synthetic construct
DIQMIQSQKFMSTSVGDRVTVTCKASQNVGTNVAWYQQKPGQSPNALIYNASHRYSGVPDRFTGSGSGTDFTLTITNVQSEDLADYFCQQYNSYPLTFGTGTKLDLK

SEQ ID NO: 14 - protein, 119 aa, synthetic construct
VQLQQSGAELVKPGASVKLSCKASGYTFTNYFIHWVKQRPGQGLEWIGEINPRNGDTDFNEKFESRATLTVDKSSSTAYMQLSSLTSEDSAIYYCTRSPYGNNYGFTYWGQGTLVTVSA

SEQ ID NO: 15 - protein, 119 aa, synthetic construct
VQLQQSGAELVKPGASVKLSCKASGYTFTNYFIHWVKQRPGQGLEWIGEINPKNGDTGFNEKFESRATLTVDKSSSTAYMQLSSLTSEDSAIYYCTRSPYGNNYGFTYWGPGTLVTVSA

SEQ ID NO: 16 - protein, 119 aa, synthetic construct
VQLQQSGAELVKPGASVKLSCKASGYTFTNYFIYWVKQRPGQGLEWIGEINPRNGDTDFNVKFKSRATLTVDKSSSTAYMQLSSLTSEDSAIYYCTRSPYRNNYGFTYWGQGTLVTVSA

SEQ ID NO: 17 - protein, 70 aa, Homo sapiens
EAEEDGDLQCLCVKTTSQVRPRHITSLEVIKAGPHCPTAQLIATLKNGRKICLDLQAPLYKKIIKKLLES

SEQ ID NO: 18 - protein, 11 aa, synthetic construct
KASQNVGTNVA

SEQ ID NO: 19 - protein, 7 aa, synthetic construct
SASYRYS

SEQ ID NO: 20 - protein, 9 aa, synthetic construct
QQYNSYPLT

SEQ ID NO: 21 - protein, 5 aa, synthetic construct
NYFIY

SEQ ID NO: 22 - protein, 17 aa, synthetic construct
EINPRNGDTDFNEKFES

SEQ ID NO: 23 - protein, 11 aa, synthetic construct
SPYGNNYGFTY

SEQ ID NO: 24 - protein, 5 aa, synthetic construct
KYFIY

SEQ ID NO: 25 - protein, 17 aa, synthetic construct
EINPRNGDTNFNEKFES

SEQ ID NO: 26 - protein, 7 aa, synthetic construct
NASHRYS

SEQ ID NO: 27 - protein, 5 aa, synthetic construct
NYFIH

SEQ ID NO: 28 - protein, 17 aa, synthetic construct
EINPKNGDTGFNEKFES

SEQ ID NO: 29 - protein, 17 aa, synthetic construct
EINPRNGDTDFNVKFKS

SEQ ID NO: 30 - protein, 11 aa, synthetic construct
SPYRNNYGFTY

SEQ ID NO: 31 - protein, 15 aa, synthetic construct
GGGGSGGGGSGGGGS

SEQ ID NO: 32 - DNA, 723 bp, synthetic construct
  source 1..723: mol_type = other DNA
gatatccagatgatccagagccagaaattcatgagcaccagcgtgggcgacagagtgaccgtgacatgtaaagccagccagaacgtgggcaccaacgtggcctggtatcagcagaagcctggacagagccccaacgctctgatctacagcgccagctacagatacagcggcgtgcccgatagattcacaggcagcggctctggcaccgacttcaccctgaccatcaccaatgtgcagagcgaggacctggccgactacttctgccagcagtacaacagctaccctctgacctttggcaccggcaccaagctggatcttaaaggcggcggaggatctggcggaggtggaagcggaggcggtggatctgttcagctgcaacaatctggcgccgagctggttaagcctggcgcctctgtgaagctgagctgcaaggccagcggctacaccttcaccaactacttcatctactgggtcaagcagcggccaggccagggactcgaatggatcggagagatcaaccccagaaacggcgacaccgatttcaacgagaagttcgagagccgggccacactgaccgtggataagtctagcagcaccgcctacatgcagctgagcagcctgacaagcgaggactccgccatctactactgcacaagaagcccctacggcaacaactacggctttacctattggggccagggcaccctggttaccgtgtctgcc

SEQ ID NO: 33 - DNA, 723 bp, synthetic construct
  source 1..723: mol_type = other DNA
gatatccagatgatccagagccagaaattcatgagcaccagcgtgggcgacaaagtgaccgtgacatgtaaagccagccagaacgtgggcaccaacgtggcctggtatcagcagaagcctggacagagccccaacgctctgatctacagcgccagctacagatacagcggcgtgcccgatagattcacaggcagcggctctggcaccgacttcaccctgaccatcaccaatgtgcagagcgaggacctggccgactacttctgccagcagtacaacagctaccctctgacctttggcaccggcaccaagctggatcttaaaggcggcggaggatctggcggaggtggaagcggaggcggtggatctgttcagctgcaacaatctggcgccgagctggttaagcctggcgcctctgtgaagctgagctgcaaggccagcggctacaccttcaccaagtacttcatctactgggtcaagcagcggccaggccagggactcgaatggatcggagagatcaaccccagaaacggcgacaccaatttcaacgagaagttcgagagccgggccacactgaccgtggataagtctagcagcaccgcctacatgcagctgagcagcctgacaagcgaggactccgccatctactactgcacaagaagcccctacggcaacaactacggctttacctattggggccagggcaccctggttaccgtgtctgcc

SEQ ID NO: 34 - DNA, 723 bp, synthetic construct
  source 1..723: mol_type = other DNA
gatatccagatgatccagagccagaaattcatgagcaccagcgtgggcgacagagtgaccgtgacatgtaaagccagccagaacgtgggcaccaacgtggcctggtatcagcagaagcctggacagagccccaacgctctgatctacaacgccagccacagatacagcggcgtgcccgatagattcacaggcagcggctctggcaccgacttcaccctgaccatcaccaatgtgcagagcgaggacctggccgactacttctgccagcagtacaacagctaccctctgacctttggcaccggcaccaagctggatcttaaaggcggcggaggatctggcggaggtggaagcggaggcggtggatctgttcagctgcaacaatctggcgccgagctggttaagcctggcgcctctgtgaagctgagctgcaaggccagcggctacaccttcaccaactacttcatctactgggtcaagcagcggccaggccagggactcgaatggatcggagagatcaaccccagaaacggcgacaccgatttcaacgagaagttcgagagccgggccacactgaccgtggataagtctagcagcaccgcctacatgcagctgagcagcctgacaagcgaggactccgccatctactactgcacaagaagcccctacggcaacaactacggctttacctattggggccagggcaccctggttaccgtgtctgcc

SEQ ID NO: 35 - DNA, 723 bp, synthetic construct
  source 1..723: mol_type = other DNA
gatatccagatgatccagagccagaaattcatgagcaccagcgtgggcgacagagtgaccgtgacatgtaaagccagccagaacgtgggcaccaacgtggcctggtatcagcagaagcctggacagagccccaacgctctgatctacagcgccagctacagatacagcggcgtgcccgatagattcacaggcagcggctctggcaccgatttcaccctgaccatcaccaatgtgcagagcgaggacctggccgactacttctgccagcagtacaacagctatcctctgacctttggcaccggcaccaagctggatcttaaaggcggcggaggatctggcggaggtggaagcggaggcggtggatctgttcagctgcaacaatctggcgccgagctggttaagcctggcgcctctgtgaagctgagctgcaaggccagcggctacaccttcaccaactacttcatccactgggtcaagcagcggccaggccagggactcgaatggataggagagatcaaccccagaaacggcgacaccgatttcaacgagaagttcgagagccgggccacactgaccgtggataagtctagcagcaccgcctacatgcagctgagcagcctgacaagcgaggactccgccatctactactgcacaagaagcccctacggcaacaactacggctttacctattggggccagggcaccctggttaccgtgtctgcc

SEQ ID NO: 36 - DNA, 723 bp, synthetic construct
  source 1..723: mol_type = other DNA
gatatccagatgatccagagccagaaattcatgagcaccagcgtgggcgacagagtgaccgtgacatgtaaagccagccagaacgtgggcaccaacgtggcctggtatcagcagaagcctggacagagccccaacgctctgatctacagcgccagctacagatacagcggcgtgcccgatagattcacaggcagcggctctggcaccgacttcaccctgaccatcaccaatgtgcagagcgaggacctggccgactacttctgccagcagtacaacagctaccctctgacctttggcaccggcaccaagctggatcttaaaggcggcggaggatctggcggaggtggtagcggaggcggtggatctgttcagctgcaacaatctggcgccgagctggttaagcctggcgcctctgtgaagctgagctgcaaggccagcggctacaccttcaccaactacttcatccactgggtcaagcagcggccaggccagggactcgaatggatcggagagatcaaccccaaaaacggcgacaccggtttcaacgagaagttcgagagccgggccacactgaccgtggataagtctagcagcaccgcctacatgcagctgagcagcctgacaagcgaggactccgccatctactactgcacaagaagcccctacggcaacaactacggctttacctattggggcccgggcaccctggttaccgtgtctgcc

SEQ ID NO: 37 - DNA, 723 bp, synthetic construct
  source 1..723: mol_type = other DNA
gatatccagatgatccagagccagaaattcatgagcaccagcgtgggcgacagagtgaccgtgacatgtaaagccagccagaacgtgggcaccaacgtggcctggtatcagcagaagcctggacagagccccaacgctctgatctacagcgccagctacagatacagcggcgtgcccgatagattcacaggcagcggctctggcaccgacttcaccctgaccatcaccaatgtgcagagcgaggacctggccgactacttctgccagcagtacaacagctaccctctgacctttggcaccggcaccaagctggatcttaaaggcggcggaggatctggcggaggtggaagcggaggcggtggatctgttcagctgcaacaatctggcgccgagctggttaagcctggcgcctctgtgaagctgagctgcaaggccagcggctacaccttcaccaactacttcatctactgggtcaagcagcggccaggccagggactcgaatggatcggagagatcaaccccagaaacggcgacaccgatttcaacgtgaagttcaaaagccgggccacactgaccgtggataagtctagcagcaccgcctacatgcagctgagcagcctgacaagcgaggactccgccatctactactgcacaagaagcccctaccgcaacaactacggctttacctattggggccagggcaccctggttaccgtgtctgcc

(19) United States
(12) Patent Application Publication
(10) Pub. No.: US 2025/0011413 A1
(43) Pub. Date: Jan. 9, 2025

(54) PRODUCTS AND METHODS FOR THE DIAGNOSIS AND TREATMENT OF HEPARIN-INDUCED THROMBOCYTOPENIA

(71) Applicant: McMaster University, Hamilton (CA)

(72) Inventors: Ishac Nazy, Hannon (CA); John Kelton, Dundas (CA); Donald Arnold, Hamilton (CA); Anna-Lise Bissola, Mississauga (CA); Colin Kretz, Mount Hope (CA)

(21) Appl. No.: 18/706,314

(22) PCT Filed: Nov. 3, 2022
(86) PCT No.: PCT/CA2022/051628

§ 371 Date: Apr. 30, 2024

Related U.S. Application Data
(60) Provisional application No. 63/275,098, filed on Nov. 3, 2021.

(51) Int. Cl. C07K 16/24; A61K 39/00; A61P 7/02; G01N 33/564; G01N 33/68

(52) U.S. Cl. CPC C07K 16/24; A61P 7/02; G01N 33/564; G01N 33/6854; A61K 2039/505; C07K 2317/33; C07K 2317/565; C07K 2317/622; C07K 2317/76; G01N 2800/222

(57) ABSTRACT

Provided herein are wildtype and mutant single chain variable fragments (scFvs) of the anti-PF4/heparin monoclonal antibody KKO, and variants thereof which are useful for distinguishing between platelet-activating (pathogenic) and non-activating (non-pathogenic) anti-PF4/heparin antibodies. Also provided herein are uses of the scFvs in methods for identifying patients with pathogenic anti-PF4/heparin antibodies, and for diagnosing and treating heparin-induced thrombocytopenia (HIT).

### CROSS-REFERENCE TO RELATED APPLICATIONS

This application claims the benefit of priority to U.S. Provisional Application No. 63/275,098, filed Nov. 3, 2021, the contents of which are incorporated herein by reference in their entirety.

### INCORPORATION OF SEQUENCE LISTING

A computer readable form of the Sequence Listing “3244-P66547PC00.xml” (39,806 bytes) created on Nov. 2, 2022, is herein incorporated by reference.

### FIELD

The present application relates to the field of thrombosis, and in particular, mutant single chain variable fragments for the diagnosis and treatment of heparin induced thrombocytopenia.

### BACKGROUND

Heparin-induced thrombocytopenia (HIT) is an immune-mediated adverse drug reaction to the anticoagulant heparin. HIT is characterized by pathogenic antibodies that form immune complexes with platelet factor 4 (PF4) and heparin, which causes platelet activation and thrombosis. Current HIT laboratory diagnostic assays face limitations surrounding either detection specificity or performance feasibility. Approximately 50-70% of patients exposed to heparin can produce anti-PF4/heparin antibodies depending on the clinical situation, but only a fraction of these antibodies are able to activate platelets and cause HIT.¹⁻⁶ For instance, anti-PF4/heparin antibody production is remarkably common in patients undergoing cardiopulmonary bypass surgery, but the frequency of HIT in these patients is low.⁶ Therefore, the polyclonal and polyspecific HIT immune response creates difficulties distinguishing between platelet-activating (pathogenic) and non-activating (non-pathogenic) antibodies.⁷ Although immunoassays, like the anti-PF4/heparin IgG-EIA, are easy to perform and offer high sensitivity (˜90%)⁸ detection of anti-PF4/heparin antibodies, they have a low specificities (˜50-70%)^{6,9} for platelet activating pathogenic HIT antibodies.

Low specificity tests exacerbate these diagnostic challenges as many patients referred for testing are falsely positive in immunoassays (20.9%; EIA-positive/SRA-negative).¹⁰ Although most suspected patients do not have HIT (65.6%; EIA-negative/SRA-negative),¹⁰ the reliance on immunoassays or rapid assays for diagnosis contribute to over-diagnosis and over-treatment.^{10,11} Unnecessary HIT treatments increase the risk of bleeding events, which can have detrimental consequences for patients.^{10,11} However, HIT patients experience a 5-10% daily increased risk of experiencing severe thrombotic events, which necessitates immediate treatment.^{12,13} Despite the improved diagnostic specificity provided by platelet activating assays, they are laborious, technically challenging, and can further delay diagnosis.^{9,10,14}

In Canada, only a small number of laboratories are equipped to perform HIT testing and even fewer that perform functional platelet activation assays. This can lead to longer turnaround times for referring hospitals, leading doctors to begin treatment ahead of laboratory confirmation. An more accurate and reliable test for HIT would reduce the reliance on a clinical or immunoassay diagnosis alone, which currently have poor specificity and lead to disease overall.¹⁰ Furthermore, accessible testing would allow clinicians to receive laboratory results faster without compromising diagnostic accuracy, not only improving patient clinical outcomes by implementing earlier treatment but shortening hospital stays and reducing associated costs of treatment. For instance, a global study conducted in 2016 found that speculative treatment of HIT with a replacement medication ahead of laboratory confirmation is associated with maximum total costs of $39,616, $11,839, and $6833 USD per patient in the US, UK, and Germany, respectively.¹⁵ However, availability of an accurate and rapid assay with the ability to differentiate between platelet activating (pathogenic) and non-activating (non-pathogenic) antibodies remains a key challenge when diagnosing HIT.

### SUMMARY

Previous studies of the polyclonal immune response in HIT have identified the existence of multiple antibody binding sites on PF4.^{16,17} Epitope mapping of anti-PF4/heparin antibodies have reveled several clinically significant binding sites on PF4 using HIT patient sera and the murine monoclonal antibody KKO¹⁸ as a model for pathogenic HIT antibodies (FIG. 1). This work revealed pathogenic antibodies against PF4/heparin from multiple HIT patients bind to a localized region on PF4.¹⁹ The monoclonal antibody KKO was also found to bind the same overlapping site as anti-PF4/heparin antibodies, despite having different PF4 epitopes.¹⁹ Further epitope mapping of sera containing false-positive anti-PF4/heparin antibodies (HIT-negative; EIA+/SRA−) also showed that non-pathogenic antibodies do not bind any consistent region on PF4.¹⁹ This is unlike what is observed with pathogenic HIT antibodies, suggesting a difference in their binding sites. Therefore, pathogenic antibodies recognize a specific region on PF4 that is also distinct from non-pathogenic antibody binding sites. This is further supported by previous inhibition experiments using HIT-positive and HIT-negative patient sera containing anti-PF4/heparin antibodies.^{18,20} Therefore, a useful strategy to improve the accuracy of current diagnostic assays for HIT could involve blocking this key epitope on PF4 to inhibit pathogenic antibody binding.

The present application discloses mutants of a single chain variable fragment (scFv) derived from KKO. As demonstrated herein, a KKO-derived scFv and mutants thereof disclosed herein can be used in a diagnostic assay to rapidly identify patients with pathogenic anti-PF4/heparin antibodies based on their specific binding sites on PF4. ScFvs are generated from the variable heavy and light chain Fab domains of an antibody, which allows it to retain antigen-binding functions while lacking the Fc fragment. This is an important feature of the disclosure because KKO-scFv can still bind to the pathogenic site on PF4, but unlike full-length KKO, is unable to interact with platelet Fc receptors and cause platelet activation. To improve affinity, a mutagenesis library of KKO-derived scFvs was created using error prone polymerase chain reaction (PCR). This library was cloned into the pADL-22c phage display vector (Antibody Design Labs Inc.), with a library depth of 1.5×10⁷ unique sequences. Phage displaying the mutant scFv library underwent 5 rounds of bio-panning against biotin-heparin/PF4 using streptavidin beads. After each round of bio-panning, 40-50 colonies were selected at random and analyzed by Sanger sequencing (McMaster Genomics Facility, Mobix Laboratory) to identify mutants that had become enriched. The resulting antibodies are useful for in competitive assays for identifying pathogenic anti-PF4/heparin antibodies in patient samples, and are shown to inhibit platelet activation in a modified serotonin-release assay (SRA) and are useful for treating or preventing HIT.

An aspect includes an isolated anti-PF4 antibody which specifically binds an epitope of PF4, wherein the antibody binds PF4 and/or a PF4/heparin complex with at least or about 2-fold, at least or about 3-fold, at least or about 4-fold, at least or about 5-fold, at least or about 10-fold, at least or about 100-fold, or more than 100-fold greater affinity than an scFv having an amino acid sequence of SEQ ID NO: 3 as determined by Biolayer Interferometry (BLI).

In an embodiment, the antibody comprises a light chain variable (VL) domain and a heavy chain variable (VH) domain, the VL domain comprising complementarity determining regions (CDRs) CDR-L1, CDR-L2, and CDR-L3, and the VH domain comprising CDRs CDR-H1, CDR-H2, and CDR-H3, wherein the amino acid sequences of said CDRs are as shown in of any one of a), b), c), d), or e):

a)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 24
KYFIY;
CDR-H2
SEQ ID NO: 25
EINPRNGDTNFNEKFES;
and
CDR-H3
SEQ ID NO: 23
SPYGNNYGFTY;
b)
CDR-L1
SEQ ID NO: 18
KASQNVGTNVA;
CDR-L2
SEQ ID NO: 26
NASHRYS;
CDR-L3
SEQ ID NO: 20
QQYNSYPLT;
CDR-H1
SEQ ID NO: 21
NYFIY
CDR-H2
SEQ ID NO: 22
EINPRNGDTDFNEKFES
and
CDR-H3
SEQ ID NO: 23
SPYGNNYGFTY;
c)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 22
EINPRNGDTDFNEKFES
and
CDR-H3
SEQ ID NO: 23
SPYGNNYGFTY;
d)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 28
EINPKNGDTGFNEKFES;
and
CDR-H3
SEQ ID NO: 23
SPYGNNYGFTY;
or
e)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 21
NYFIY
CDR-H2
SEQ ID NO: 29
EINPRNGDTDFNVKFKS;
and
CDR-H3
SEQ ID NO: 30
SPYRNNYGFTY.

In an embodiment, the VL domain and VH domain comprise i) a polypeptide having an amino acid sequence of a) SEQ ID NOs: 11 and 12; b) SEQ ID NOs: 13 and 10; c) SEQ ID NOs: 9 and 14; d) SEQ ID NOs: 9 and 15; or e) SEQ ID NOs: 9 and 16; ii) a polypeptide having an amino acid sequence with at least 80%, at least 90%, or at least 95% sequence identity to a) SEQ ID NOs: 11 and 12; b) SEQ ID NOs: 13 and 10; c) SEQ ID NOs: 9 and 14; d) SEQ ID NOs: 9 and 15; or e) SEQ ID NOs: 9 and 16 wherein the CDR sequences are those described herein; or iii) a conservatively substituted amino acid sequence of i) wherein the CDR sequences are those described herein.

In an embodiment, the VL domain comprises i) a polypeptide having an amino acid sequence of SEQ ID NO: 9; ii) a polypeptide having an amino acid sequence with at least 80%, at least 90%, or at least 95% sequence identity to SEQ ID NO: 9; or iii) a conservatively substituted amino acid sequence of SEQ ID NO: 9, and the VH domain comprises i) a polypeptide having an amino acid sequence of SEQ ID NO: 10; ii) a polypeptide having an amino acid sequence with at least 80%, at least 90%, or at least 95% sequence identity to SEQ ID NO: 10; or iii) a conservatively substituted amino acid sequence of SEQ ID NO: 10, and wherein the antibody comprises one or more mutations at positions selected from R18, S50, and Y53 of SEQ ID NO: 9, and/or one or more mutations at positions selected from N30, Y34, D58, E61, E64, G101, and Q111 of SEQ ID NO: 10.

In an embodiment, the one or more mutations are selected from R18K, S50N, and Y53H of SEQ ID NO: 9, and/or selected from N30K, Y34H, D58N, D58G, E61V, E64K, G101R, and Q111P of SEQ ID NO: 10.

In an embodiment, the one or more mutations are selected from the following combinations a), b), c), d), and e):
a) R18K of SEQ ID NO: 9 and N30K and D58N of SEQ ID NO: 10;
b) S50N and Y53H of SEQ ID NO: 9;
c) Y34H of SEQ ID NO: 10;
d) Y34H, D58G, and Q111P of SEQ ID NO: 10; and
e) E61V, E64K, and G101R of SEQ ID NO: 10.

In an embodiment the antibody is an antibody fragment that does not comprise an Fc domain.

In an embodiment, the antibody is a scFv.

In an embodiment, the scFv comprises, from N-terminus to C-terminus, VL-linker-VH.

In an embodiment, the scFv comprises a polypeptide having an amino acid sequence of any one of SEQ ID NOs: 4-8.

An aspect includes a nucleic acid molecule encoding the antibody or fragment thereof described herein.

In an embodiment, the nucleic acid molecule has a sequence of any one of SEQ ID NOs: 33-37, or functional variants thereof.

An aspect includes a cell comprising a nucleic acid molecule described herein, or expressing an antibody or fragment thereof described herein.

An aspect includes a pharmaceutical composition comprising the antibody or fragment thereof described herein and a pharmaceutically acceptable carrier or excipient.

An aspect includes a method of diagnosing heparin-induced thrombocytopenia (HIT) in a patient, the method comprising: a) obtaining a biological sample comprising patient antibodies to PF4/heparin from the patient; b) contacting the sample with i) PF4/heparin in the presence of an antibody or fragment thereof described herein, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, and ii) PF4/heparin in the absence of an antibody described herein, or a wildtype KKO antibody fragment, under conditions permissive for forming PF4/heparin:patient antibody complexes; c) detecting the presence of any PF4/heparin:patient antibody complexes in i) and ii), wherein the detecting does not detect the antibody or fragment thereof described herein, or the wildtype KKO antibody fragment; and d) determining the relative amount of PF4/heparin:patient antibody complexes in i) and ii) thereby determining if PF4/heparin:patient antibody binding is inhibited; wherein the patient is diagnosed as having HIT if PF4/heparin:patient antibody binding is inhibited.

In an embodiment, the PF4/heparin is contacted with the antibody or fragment thereof described herein, or the wildtype KKO antibody fragment prior to contacting with the sample.

In an embodiment, the biological sample comprises blood, serum, or plasma.

An aspect includes an antibody or fragment thereof described herein, a pharmaceutical composition comprising said antibody or fragment thereof, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, for use in the treatment or prevention of heparin-induced thrombocytopenia in a subject in need thereof.

An aspect includes a use of an antibody or fragment thereof described herein, a pharmaceutical composition comprising said antibody or fragment thereof, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, for the treatment or prevention of heparin-induced thrombocytopenia in a subject in need thereof.

An aspect includes a use of an antibody or fragment thereof described herein, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, in the manufacture of a medicament for the treatment or prevention of heparin-induced thrombocytopenia.

An aspect includes a method of treating or preventing heparin-induced thrombocytopenia, the method comprising administering a therapeutically effective amount of an antibody or fragment thereof described herein, a pharmaceutical composition comprising said antibody or fragment thereof, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, to a subject in need thereof.

Other features and advantages of the present application will become apparent from the following detailed description. It should be understood, however, that the detailed description and the specific examples, while indicating embodiments of the application, are given by way of illustration only and the scope of the claims should not be limited by these embodiments but should be given the broadest interpretation consistent with the description as a whole.

### DRAWINGS

The embodiments of the application will now be described in greater detail with reference to the attached drawings in which:

FIG. 1 shows sequences of wildtype (SEQ ID NO: 3) and mutant scFvs B-F (SEQ ID NOs: 4-8, respectively) in an exemplary embodiment of the application. Lead mutant candidates were selected after five rounds of phage display bio-panning based on frequency. Asterisks (*) indicate conserved residues. Sequence alignment, analysis, and figure were generated using Clustal Omega version 1.2.4.

FIG. 2 shows the purification of wildtype and mutant scFv from BL21 cells using Ni-NTA affinity chromatography. SDS-PAGE and Coomassie Simply Blue™ SafeStain analysis of NI-NTA column purification of scFv wildtype and mutant constructs. Std represents protein ladder standards (kDa). Lanes 1-6 represent concentrated eluates containing scFv displaced from the column at 500 mM imidazole buffer. Lane 1 shows wildtype scFv, lane 2 shows scFv mutant B, lane 3 shows scFv mutant C, lane 4 shows scFv mutant D, lane 5 shows scFv mutant E, and lane 6 shows scFv mutant F. The arrow indicates protein bands corresponding to purified scFv variants at approximately 28 kDa. All samples were separated on a denaturing 4-20% SDS polyacrylamide gradient gel followed by Coomassie staining.

FIG. 3 shows binding kinetics of wildtype scFv against biotinylated PF4 complexes without heparin immobilized on streptavidin sensors in an exemplary embodiment of the application. a) Spectrogram showing the binding response of wildtype scFv with lines of best fit. Binding was measured at six different concentrations: 4 μg/mL, 2 μg/mL, 1 μg/mL, 0.5 μg/mL, and 0.25 μg/mL. b) Table showing BLI binding responses and kinetic data, including binding affinity (K_{D}) as well as association (kₒₙ) and dissociation (k_{off}) rates. All data was analyzed based on a 1:1 homogenous ligand binding model using Octet® User Software version 3.1.

FIG. 4 shows binding kinetics of wildtype scFv against biotinylated PF4 and heparin complexes (PF4/heparin) immobilized on streptavidin sensors in an exemplary embodiment of the application. a) Spectrogram representing the binding response of wildtype scFv with lines of best fit. Binding was measured at six different concentrations: 4 μg/mL, 2 μg/mL, 1 μg/mL, 0.5 μg/mL, and 0.25 μg/mL. b) Table showing BLI binding responses and kinetic data, including association (kₒₙ) and dissociation (k_{off}) rate constants and binding affinity (K_{D}). All data was analyzed based on a 1:1 homogenous ligand binding model using Octet®) User Software version 3.1.

FIG. 5 shows BLI Binding response of mutant scFv against biotinylated PF4 complexes without heparin in an exemplary embodiment of the application. a) Spectrogram representing the binding responses of wildtype and mutant scFv showing lines of best fit. BLI experiments were performed using wildtype scFv, mutant B, mutant C, mutant D, mutant E, and mutant F at 1 μg/mL. Binding was measured against immobilized biotinylated PF4 on streptavidin biosensors. b) Table showing kinetic data of wildtype and mutant scFv, including binding responses and association and dissociation rates. All data was analyzed based on a 1:1 homogenous ligand binding model using Octet® User Software version 3.1.

FIG. 6 shows BLI binding response of mutant scFv against PF4/heparin in an exemplary embodiment of the application. a) Spectrogram representing the binding responses of wildtype and mutant scFv showing lines of best fit. BLI experiments were performed using wildtype scFv, mutant B, mutant C, mutant D, mutant E, and mutant F at 1 μg/mL. Binding was measured against immobilized biotinylated PF4/heparin complexes on streptavidin biosensors. b) Table showing kinetic data of wildtype and mutant scFv, including binding responses and association and dissociation rates. All data was analyzed based on a 1:1 homogenous ligand binding model using Octet®) User Software version 3.1.

FIG. 7 shows wildtype scFv inhibits full-length KKO in an exemplary embodiment of the application. Dose-dependent inhibition of KKO (2 μg/mL) from binding to PF4/heparin complexes using concentrations of scFv ranging from 0 to 160 μg/mL (n=3). Results are shown as the percentage (%) of KKO binding in the presence of wildtype, mutant B, mutant C, mutant D, mutant E, and mutant F scFv at increasing concentrations. The ability of each construct to inhibit KKO binding resulting in a >50% decrease (black dotted line) in absorbance (OD) at 405 nm was determined relative to control wells containing KKO in the absence of scFv.

FIG. 8 shows the amino acids that are critical for the binding of scFv to PF4. a) PF4 tetramer showing full-length KKO, wildtype scFv, scFv mutant B, scFv mutant C, scFv mutant D, scFv mutant E, and scFv mutant F surface antibody binding sites. b) The primary sequence of PF4 (SEQ ID NO: 17) highlighting amino acids predicted to make-up the binding sites for scFv wildtype, scFv mutant B, scFv mutant C, scFv mutant D, scFv mutant E, and scFv mutant compared to full-length (FL) KKO. Images are modified from the Protein Data Bank (PDB) entry 1RHP.

FIG. 9 shows the large-scale screening of HIT-positive and HIT-negative patients in a streptavidin enzyme immunoassay (EIA) using scFv wildtype, mutant B, and mutant F. Previously tested HIT patient samples (HIT positive sera, n=20; HIT negative sera, n=20) were tested in an anti-PF4 IgG-specific streptavidin inhibition EIA. Results are shown as the absorbance (OD) at 405 nm with (+scFv) or without (−scFv) the addition of the indicated scFv or mutant variant. The ability of wildtype, mutant B or mutant F scFv to inhibit antibody binding resulting in a decrease in absorbance below the negative cut-off (OD₄₀₅ₙₘ=0.45, dotted line) was determined. Statistical significance was calculated using an unpaired t-test where (***) represents p>0.0005.

FIG. 10 shows how this assay can distinguish between pathogenic and non-pathogenic anti-PF4/heparin antibodies in a streptavidin EIA. Previously tested EIA-positive/SRA-positive HIT patient samples (HIT positive sera; n=20) and EIA-positive/SRA-negative (HIT negative sera; n=20) were tested in a modified streptavidin EIA. Results are shown as the percent (%) inhibition of anti-PF4/heparin antibody binding in the presence of scFv wildtype, mutant B, or mutant F at 50 μg/mL. The ability of each scFv construct to inhibit antibody binding resulting in a decrease in binding below 50% (dotted line) was determined. Statistical significance was calculated using a two-way ANOVA analysis where (***) represents p>0.0005 and (****) represents p<0.00005. Error bars denoting mean inhibition±standard deviation (s.d.) are shown.

FIG. 11 shows the receiver operating characteristics (ROC) curves of an IgG-specific streptavidin anti-PF4/heparin EIA using scFv. ROC curves were generated for the PF4/heparin IgG-specific streptavidin EIA alone and using scFv wildtype, mutant B, or mutant obtained from testing two clinical cohorts of HIT-positive (n=20, EIA+/SRA+) and HIT-negative (n=20, EIA+/SRA−).

FIG. 12 shows inhibition of HIT antibody-mediated platelet activation in the ¹⁴C-serotonin release assay by scFv. Previously tested EIA-positive/SRA-positive HIT patient samples (n=5) were tested in a modified ¹⁴C-serotonin-release assay (SRA) in the presence of scFv a) wildtype, b) mutant B, c) mutant C, d) mutant D, e) mutant E, or f) mutant F at either 0, 50, or 75 μg/mL concentrations. The ability of each scFv construct to inhibit platelet activation resulting in a moderate (≥20%) or strong (≥50%) decrease in percent ¹⁴C-serotonin release was determined relative to control wells containing patient sera in the absence of scFv.

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

HIT is an antibody-mediated drug disorder arising in patients receiving heparin as an anticoagulant medication, often following major surgical procedures.^{6,21} Heparin binds favourably to PF4 tetramers and forms structurally stabilized complexes, which can trigger anti-PF4/heparin antibody production and lead to the formation of large immune complexes.²¹⁻²⁵ Although the development of anti-PF4/heparin antibodies is common in heparin-treated patients, only a small percentage of individuals develop HIT.^{1,6,25-27} This is because PF4/heparin induces a highly polyclonal and polyspecific antibody response where most antibodies produced are non-pathogenic and cannot cause HIT.^{7,10,28} Rapid and accurate differentiation between anti-PF4/heparin antibodies that can and cannot cause platelet activation remains a significant diagnostic challenge due to the reduced specificity and limited availability of current laboratory assays.^{10,11} Recently, it was shown that pathogenic antibodies bind to a restricted region on PF4 distinct from sites bound by non-pathogenic antibodies.¹⁹ Based on these different binding sites, this work aims to use an epitope-targeted strategy to distinguish between pathogenic and non-pathogenic antibodies in an EIA using scFv mutants by blocking the heparin-dependent pathogenic epitope on PF4.

In the present disclosure, random mutagenesis was used to improve the affinity of an scFv derived from the monoclonal antibody KKO. Five mutant variants of scFv were identified using phage display after various rounds of bio-panning selection on magnetic beads coated with the target antigen for HIT antibodies, PF4/heparin. Functional studies were then carried out to assess the binding characteristics and affinity of each variant compared to wildtype. BLI was used for kinetic analysis of wildtype and mutant scFv. Mutants B, D, E, and F had substantially improved affinity towards PF4 alone and PF4 complexed with heparin compared to wildtype, and mutant C had moderately improved affinity towards PF4/heparin compared to wildtype. Binding inhibition experiments using KKO were also performed to evaluate the strength of each construct against a full-length antibody and determine approximate IC50 values. These studies revealed that scFv mutants B, D, E, and F were able to strongly inhibit KKO binding in a streptavidin anti-PF4/hep EIA.

As further shown in the Examples, epitope mapping revealed scFv wildtype, mutant C, and mutant D bound to the same amino acids as full-length KKO. Whereas mutant B recognized amino acids that differed from the other variants but still overlapped with the heparin-dependent binding site on PF4. A small sample of patient sera was then tested in an anti-PF4/heparin EIA using wildtype and mutant scFv, which showed each construct inhibited HIT-positive antibodies but did not affect the binding of HIT-negative antibodies. These findings suggest scFv can eliminate false-positive signals in an EIA that arise from antibodies that do not cause HIT.

As shown in the Examples, wildtype scFv and each of the five mutants reduced antibody binding from HIT-positive patients. Four scFv constructs, mutants B, D, E, and F, were able to reduce the false-positive rate of EIAs caused by the presence of non-pathogenic antibodies when testing HIT-positive and HIT-negative patient sera. Pathogenic antibody binding was reduced to negative detection levels (OD₄₀₅ₙₘ<0.45) in this optimized assay in the presence of mutant B, D, E, and F. As predicted, these four mutants also had a stronger effect and higher mean percent inhibition of pathogenic antibody binding compared to both wildtype and mutant C (Table 5-6). These findings also show a correlation with BLI analysis and inhibition studies with KKO, which demonstrate mutant B, D, E, and F have superior performance and binding kinetics. Out of five mutant scFv constructs, two were chosen as lead candidates to move forward with to perform a large-scale screening of patient sera and evaluate the diagnostic performance of this assay.

Mutant B and F demonstrated the foremost ability to distinguish between pathogenic and non-pathogenic antibodies compared to all scFv constructs. Testing these mutants against a larger population of HIT-positive (n=20) and HIT-negative (n=20) patient samples revealed pathogenic antibody binding was almost always significantly reduced in the presence of scFv while non-pathogenic antibody binding was not. ROC curve analysis revealed a significant increase in the performance of the streptavidin anti-PF4/hep EIA with the addition of scFv. Compared to previous literature reports of the anti-PF4/hep EIA diagnostic performance, 8.9 incorporating scFv showed an improvement in specificity to 90.0% while maintaining the high sensitivity of this assay 29.30 Overall, targeting clinically significant epitopes on PF4 proves to be an effective method of distinguishing between HIT antibodies based on their individual binding sites. The addition of wildtype or mutant scFv also demonstrates improved diagnostic performance compared to the IgG-specific anti-PF4/heparin EIA for identifying clinically significant HIT antibodies.

In the present disclosure, scFv mutants demonstrated an advanced diagnostic performance in EIAs used to identify HIT antibodies. Current EIAs designed to diagnose HIT have low specificities (˜50-70%)^{6,9} for clinically significant antibodies in HIT. Automated rapid assays are also frequently employed for HIT diagnosis,³¹⁻³⁵ but they can require costly and specialized equipment to perform, making them inaccessible to many labs despite their higher specificities. The addition of mutant B and F scFv to the streptavidin anti-PF4/hep EIA improved the diagnostic specificity to 90.0% in a cohort containing HIT-positive and HIT-negative patients without reducing sensitivity. The assay described here provides a promising alternative to functional and automated rapid assays for the diagnosis of HIT. High affinity scFv mutants can accurately differentiate between pathogenic and non-pathogenic HIT antibodies in an EIA, thus providing a rapid and cost-effective solution to common limitations of HIT diagnostic assays. Furthermore, this EIA can be performed easily with standard laboratory equipment, increasing the overall availability of HIT testing.

Unless otherwise indicated, the definitions and embodiments described in this and other sections are intended to be applicable to all embodiments and aspects of the present application herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description.

All numerical values herein are modified by “about” or “approximately” the indicated value, and take into account experimental error and variations that would be expected by a person having ordinary skill in the art.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used herein, the singular forms “a”, “an”, and “the” include plural references unless the context clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of’ or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having.” “containing,” “involving,” “holding,” “composed of,” and the like are to be understood to inclusive or be open-ended, i.e., to mean including but not limited to, and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

As used herein, the phrase “at least one,” in reference to a list of one or more elements, should be understood to mean at least one element selected from anyone or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### II. Antibodies, Nucleic Acids, and Cells

The inventors show herein the development of five mutants of a single chain variable fragment (scFv) antibodies derived from the murine KKO (referred to herein as “KKO-scFv” or “scFv”) antibody from a library of 1.5×10⁷ unique sequences. These scFv mutants bind to PF4 (SEQ ID NO: 17) and/or a PF4/heparin complex with at least 2-fold, at least 3-fold, at least 4-fold, at least 5-fold, at least 10-fold, at least 100-fold, or greater than 100-fold binding affinity relative to wildtype scFv (SEQ ID NO: 3) as determined by Biolayer Interferometry (BLI). Accordingly, provided herein are anti-PF4 antibodies which specifically bind an epitope of PF4, wherein the antibodies bind PF4 and/or a PF4/heparin complex with at least or about 2-fold, at least or about 3-fold, at least or about 4-fold, at least or about 5-fold, at least or about 10-fold, at least or about 100-fold, or more than 100-fold greater affinity than an scFv having an amino acid sequence of SEQ ID NO: 3. As used herein, “PF4/heparin” or “PF4/heparin complex” refers to multimeric protein complex comprising PF4 and heparin. Optionally, the PF4 has an amino acid sequence of SEQ ID NO: 17. The anti-PF4 antibodies described herein include antibodies comprising the complementarity determining regions (CDRs) of any one of a), b), c), d), or e):

a)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 24
KYFIY;
CDR-H2
SEQ ID NO: 25
EINPRNGDTNFNEKFES;
and
CDR-H3
SEQ ID NO: 23
SPYGNNYGFTY;
b)
CDR-L1
SEQ ID NO: 18
KASQNVGTNVA;
CDR-L2
SEQ ID NO: 26
NASHRYS;
CDR-L3
SEQ ID NO: 20
QQYNSYPLT;
CDR-H1
SEQ ID NO: 21
NYFIY
CDR-H2
SEQ ID NO: 22
EINPRNGDTDFNEKFES
and
CDR-H3
SEQ ID NO: 23
SPYGNNYGFTY;
c)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 22
EINPRNGDTDFNEKFES
and
CDR-H3
SEQ ID NO: 23
SPYGNNYGFTY;
d)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 28
EINPKNGDTGFNEKFES;
and
CDR-H3
SEQ ID NO: 23
SPYGNNYGFTY;
or
e)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 21
NYFIY
CDR-H2
SEQ ID NO: 29
EINPRNGDTDFNVKFKS;
and
CDR-H3
SEQ ID NO: 30
SPYRNNYGFTY.

The antibodies may comprise light chain variable (VL) and heavy chain variable (VH) domains set out in a) SEQ ID NOs: 11 and 12; b) SEQ ID NOs: 13 and 10; c) SEQ ID NOs: 9 and 14; d) SEQ ID NOs: 9 and 15; or e) SEQ ID NOs: 9 and 16, or variants thereof having the CDR sequences specified in a), b), c), d), or e), above.

The antibodies may comprise variants of the VL and/or VH domains of SEQ ID NOs: 9 and 10, respectively, comprising one or more mutations at positions selected from R18, S50, and Y53 of SEQ ID NO: 9, and/or one or more mutations at positions selected from N30, Y34, D58, E61, E64, G101, and Q111 of SEQ ID NO: 10, or variants thereof having the specified mutations. In an embodiment, the one or more mutations are selected from R18K, S50N, and Y53H of SEQ ID NO: 9, and/or selected from N30K, Y34H, D58N, D58G, E61V, E64K, G101R, and Q111P of SEQ ID NO: 10. In an embodiment, the one or more mutations are selected from the following combinations: a) R18K of SEQ ID NO: 9 and N30K and D58N of SEQ ID NO: 10; b) S50N and Y53H of SEQ ID NO: 9; c) Y34H of SEQ ID NO: 10; d) Y34H, D58G, and Q111P of SEQ ID NO: 10; and e) E61V, E64K, and G101R of SEQ ID NO: 10.

The basic antibody structural unit is known to comprise a tetramer composed of two identical pairs of polypeptide chains, each pair having one light (“L”) (about 25 kDa) and one heavy (“H”) chain (about 50-70 kDa). The amino-terminal portion of the light chain forms a light chain variable domain (VL) and the amino-terminal portion of the heavy chain forms a heavy chain variable domain (VH). Together, the VH and VL domains form the antibody variable region (Fv) which is primarily responsible for antigen recognition/binding. Within each of the VH and VL domains are three hypervariable regions or complementarity determining regions (CDRs, commonly denoted CDR-H1, CDR-H2, CDR-H3, CDR-L1, CDR-L2, and CDR-L3). The carboxy-terminal portions of the heavy and light chains together form a constant region (Fc domain) primarily responsible for effector function.

The term “antibody” as used herein is intended to include monoclonal antibodies, chimeric and humanized antibodies, and binding fragments thereof, including for example a single chain Fab fragment, Fab′2 fragment, or single chain Fv fragment. The antibody may be from recombinant sources and/or produced in transgenic animals. Humanized or other chimeric antibodies may include sequences from one or more than one isotype, class, or species. Antibodies may be any class of immunoglobulins including: IgG, IgM, IgD, IgA, or IgE; and any isotype thereof, including IgG1, IgG2 (e.g. IgG2a, IgG2b), IgG3 and IgG4. Further, these antibodies are typically produced as antigen binding fragments such as Fab, Fab′ F(ab′)2, Fd, Fv and single domain antibody fragments, or as single chain antibodies (e.g. scFv) in which the heavy and light chains are linked by a spacer or linker. The antibodies may include sequences from any suitable species including human. Also, the antibodies may exist in monomeric or polymeric form.

The term “antibody fragment” or “binding fragment” as used herein is intended to include without limitations Fab, Fab′, F(ab′)2, scFab, scFv, dsFv, ds-scFv, dimers, minibodies, diabodies, and multimers thereof, and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, and other fragments can also be synthesized by recombinant techniques. In some embodiments, the antibody fragment does not comprise an Fc domain and/or does not interact with platelet Fc receptors. In an embodiment, the antibody is an scFv. In an embodiment, the scFv comprises, from N-terminus to C-terminus, VL-linker-VH. In an embodiment, the linker comprises a GS-linker peptide, optionally having a sequence of SEQ ID NO: 31. In an embodiment, the scFv comprises a polypeptide having an amino acid sequence of any one of SEQ ID NOs: 4-8.

The term “complementarity determining region” or “CDR” as used herein refers to particular hypervariable regions of antibodies that are commonly presumed to contribute to epitope binding. Computational methods for identifying CDR sequences include Kabat, Chothia, and IMGT. The CDRs listed in the present disclosure are identified using Kabat. A person skilled in the art having regard to the sequences comprised herein would also be able to identify CDR sequences based on IMGT and Chothia etc. Such antibodies are similarly encompassed.

The phrase “isolated antibody” refers to antibody produced in vivo or in vitro that has been removed from the source that produced the antibody, for example, an animal, hybridoma or other cell line (such as recombinant insect, yeast or bacteria cells that produce antibody). In some embodiments the antibody is an isolated antibody. The isolated antibody is optionally “purified”, which means at least: 80%, 85%, 90%, 95%, 98% or 99% purity.

The term “epitope” as commonly used means an antibody binding site, typically a polypeptide segment having a particular structural conformation, in an antigen that is specifically recognized by the antibody. For example an antibody generated or selected against a recombinant protein comprising the identified target region (e.g. PF4; SEQ ID NO: 17) recognizes part or all of said epitope sequence.

The term “greater affinity” as used herein refers to a relative degree of antibody binding where an antibody X binds to target Y more strongly (Kₒₙ) and/or with a smaller dissociation constant (K_{off}) than does comparator antibody Z, and in this context antibody X has a greater affinity for target Y than Z. Likewise, the term “lesser affinity” herein refers to a degree of antibody binding where an antibody X binds to target Y less strongly and/or with a larger dissociation constant than does antibody Z, and in this context antibody X has a lesser affinity for target Y than Z. The affinity of binding between an antibody and its target antigen can be expressed quantitatively as KA equal to 1/K_{D} where K_{D} is equal to kₒₙ/k_{off}. As such, a greater affinity corresponds to a lower K_{D}. The kₒₙ and k_{off} values can be measured using surface plasmon resonance technology, and/or as described herein. Binding affinity can also be assessed using other techniques such as flow cytometry.

The term “functional variant” as used herein includes modifications of the polypeptide sequences disclosed herein that perform substantially the same function as the polypeptide molecules disclosed herein in substantially the same way. For example, the functional variant may comprise sequences having at least 80%, or at least 90%, or at least 95% sequence identity to the sequences disclosed herein provided that the variant retains at least or about the same binding affinity for PF4 and/or PF4/heparin. The functional variant may also comprise conservatively substituted amino acid sequences of the sequences disclosed herein.

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g. gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g. for score=100, wordlength=12 to obtain nucleotide sequences homologous to a nucleic acid molecules of the present disclosure. BLAST protein searches can be performed with the XBLAST program parameters set, e.g. to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g. of XBLAST and NBLAST) can be used (see, e.g. the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

For antibodies, percentage sequence identities can be determined when antibody sequences are maximally aligned by IMGT or other (e.g. Kabat or Chothia numbering conventions). The terms “IMGT numbering” or “ImMunoGeneTics database numbering”, which are recognized in the art, refer to a system of numbering amino acid residues which are more variable (i.e. hypervariable) than other amino acid residues in the heavy and light chain variable regions of an antibody, or antigen binding portion thereof. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage. Accordingly, IMGT and other alignment systems can also be used to identify or annotate CDRs in an antibody sequence.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

The antibodies described herein may be provided as immunoconjugates. Accordingly, also provided herein are immunoconjugates comprising an antibody described herein and a suitable reagent such as a therapeutic, cytotoxic agent, or detectable label. Suitable reagents can be identified by the skilled person depending on the application. Detectable labels include radionuclides, fluorescent dyes, enzymes, or biotin may be used depending on the application, and are contemplated herein.

Immunoconjugates may be generated using any suitable technique. Common conjugation techniques include N-hydroxysuccinimide ester (NHS ester) or maleimide crosslinking, but other techniques are known in the art.

A further aspect is an isolated nucleic acid encoding an antibody or fragment thereof described herein.

Nucleic acids encoding a heavy chain or a light chain or parts thereof are also provided, for example encoding a heavy chain variable domain comprising CDR-H1, CDR-H2 and/or CDR-H3 regions described herein or encoding a light chain variable domain comprising CDR-L1, CDR-L2 and/or CDR-L3 regions described herein, variable heavy and light domains described herein, and codon optimized and codon degenerate versions thereof.

The present disclosure also provides variants of the nucleic acid sequences that encode for the antibody and/or binding fragment thereof disclosed herein. For example, the variants include nucleotide sequences that hybridize to the nucleic acid sequences encoding the antibody and/or binding fragment thereof disclosed herein under at least moderately stringent hybridization conditions or codon degenerate or optimized sequences In another embodiment, the variant nucleic acid sequences have at least 50%, at least 60%, at least 70%, most preferably at least 80%, even more preferably at least 90% and even most preferably at least 95% sequence identity to nucleic acid sequences encoding any of the amino acid sequences described herein for example as shown in SEQ ID NOs: 4-16, or functional variants thereof.

A further aspect is an isolated nucleic acid encoding an antibody described herein, for example the nucleic acids shown in any of SEQ ID NOs: 33-37, or variants thereof.

Another aspect is an expression cassette, plasmid, or vector comprising the nucleic acid herein disclosed.

The term “expression cassette” refers to a DNA molecule encoding an RNA or protein operably linked to a promoter and a transcriptional termination signal (e.g. polyadenylation signal), such that certain portions of the expression cassette are capable of being transcribed into RNA such as a messenger RNA that is subsequently translated into protein by cellular machinery.

The term “operably linked” as used herein refers to a relationship between two components that allows them to function in an intended manner. For example, where a DNA encoding an RNA of interest is operably linked to a promoter, the promoter actuates expression of the RNA encoded therein.

The term “promoter” or “promoter sequence” generally refers to a regulatory DNA sequence capable of being bound by an RNA polymerase to initiate transcription of a downstream (i.e. 3′) sequence to generate an RNA. Suitable promoters may be derived from any organism and may be bound or recognized by any RNA polymerase. Suitable promoters will be known to the skilled person. In some expression cassettes, the promoter is an inducible promoter and/or comprises a binding sequence for a transactivator or a repressor that will activate or inhibit transcription respectively, for example IPTG-inducible promoters are commonly used for expression in E. coli. Other suitable promoters will depend on the expression system and/or host cell being used.

Suitable regulatory sequences may be derived from a variety of sources, including bacterial, fungal, viral, mammalian, or insect genes.

Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The nucleic acid molecules may be incorporated in a known manner into an appropriate expression vector which ensures expression of the protein. Possible expression vectors include but are not limited to cosmids, plasmids, or modified viruses (e.g. replication defective retroviruses, adenoviruses and adeno-associated viruses). The vector should be compatible with the host cell used. The expression vectors are “suitable for transformation of a host cell”, which means that the expression vectors contain a nucleic acid molecule encoding the peptides corresponding to antibodies described herein.

The vector can be any vector, including vectors suitable for producing an antibody and/or binding fragment thereof described herein. In an embodiment, the vector is an isolated vector.

The recombinant expression vectors may also contain a marker gene which facilitates the selection of host cells transformed, infected or transfected with a vector for expressing an antibody or epitope peptide described herein.

The recombinant expression vectors may also contain expression cassettes which encode a fusion moiety (i.e. a “fusion protein”) which provides increased expression or stability of the recombinant peptide; increased solubility of the recombinant peptide; and aid in the purification of the target recombinant peptide by acting as a ligand in affinity purification, including for example tags and labels described herein. Further, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMAL (New England Biolabs, Beverly, MA) and pRIT5 (Pharmacia, Piscataway, NJ) which fuse glutathione S-transferase (GST), maltose E binding protein, or protein A, respectively, to the recombinant protein.

Also provided in another aspect is a cell, optionally an isolated and/or recombinant cell, expressing an antibody described herein or comprising a vector herein disclosed.

The recombinant cell can be generated using any cell suitable for producing a polypeptide, for example suitable for producing an antibody and/or binding fragment thereof. For example to introduce a nucleic acid (e.g. a vector) into a cell, the cell may be transfected, transformed or infected, depending upon the vector employed.

Suitable host cells include a wide variety of prokaryotic and eukaryotic host cells. For example, the proteins described herein may be expressed in bacterial cells such as E. coli, insect cells (using baculovirus), yeast cells, or mammalian cells.

### III. Compositions

The antibodies or fragments thereof described herein are suitably formulated in a conventional manner into compositions using one or more carriers or diluents. Accordingly, the present description also includes a composition comprising one or more antibodies or fragments thereof described herein and a carrier or diluent. The antibodies or fragments thereof described herein are suitably formulated into pharmaceutical compositions or dosage forms for administration to patients in a biologically compatible form suitable for administration in vivo. Accordingly, the present description further includes a pharmaceutical composition comprising an antibody or fragment thereof described herein, and a pharmaceutically acceptable carrier. Also provided herein are dosage forms comprising an antibody or fragment thereof described herein. In some embodiments the pharmaceutical compositions or dosage forms are used in the treatment of any of the diseases, disorders or conditions described herein, for example HIT.

The term “dosage form” as used herein refers to the physical form of a dose for example comprising an antibody or fragment thereof described herein, and includes without limitation injectable dosage forms, including, for example, sterile solutions and sterile powders for reconstitution, and the like, that are suitably formulated for injection, resuspendable powders, liquids and solutions. For example the injectable dosage form can be a subcutaneous, intradermal, or intramuscular depot injection that allows the compound to be released in a controlled and consistent way over a period of time, for example over one month. Methods for making depot injections are described, for example, in U.S. Pat. No. 3,089,815 entitled “Injectable pharmaceutical preparation, and a method of making same” and herein incorporated by reference in its entirety.

The compositions or dosage forms described herein can be prepared by per se known methods for the preparation of pharmaceutically acceptable compositions that can be administered to patients, such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), dioleoylphosphatidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol,

In an embodiment, the composition comprises an antibody described herein. In another embodiment, the composition comprises an antibody described herein and a diluent. In an embodiment, the composition is a sterile composition.

The antibodies or fragments thereof described herein may be administered to a patient in a variety of forms depending on the selected route of administration, as will be understood by those skilled in the art. For example, the antibodies or fragments thereof described herein may be administered by parenteral administration and the pharmaceutical compositions formulated accordingly. In some embodiments, administration is by means of a pump for periodic or continuous delivery. Conventional procedures and ingredients for the selection and preparation of suitable compositions are described, for example, in Remington's Pharmaceutical Sciences (2000—20th edition) and in The United States Pharmacopeia: The National Formulary (USP 24 NF 19) published in 1999.

Parenteral administration includes systemic delivery routes other than the gastrointestinal (GI) tract, and includes, for example intravenous, intra-arterial, intraperitoneal, subcutaneous, intramuscular, modes of administration. Parenteral administration may be by continuous infusion over a selected period of time.

In some embodiments, the antibodies or fragments thereof described herein are administered parenterally. For example, solutions of one or more antibodies or fragments thereof described herein are prepared in water suitably mixed with a surfactant such as hydroxypropylcellulose. In some embodiments, dispersions are prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the antibodies or fragments thereof described herein are usually prepared, and the pH of the solutions are suitably adjusted and buffered. For intravenous use, the total concentration of solutes should be controlled to render the preparation isotonic. For ocular administration, ointments or droppable liquids are delivered, for example, by ocular delivery systems known to the art such as applicators or eye droppers. In some embodiment, such compositions include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

In some embodiments, the antibodies or fragments thereof described herein are formulated for parenteral administration by injection, including using conventional catheterization techniques or infusion. Formulations for injection are, for example, presented in unit dosage form, e.g., in ampoules or in multi-dose containers, with an added preservative. In some embodiments, the compositions take such forms as sterile suspensions, solutions or emulsions in oily or aqueous vehicles, and contain formulating agents such as suspending, stabilizing and/or dispersing agents. In all cases, the form must be sterile and must be fluid to the extent that easy syringability exists. Alternatively, antibodies or fragments thereof described herein are suitably in a sterile powder form for reconstitution with a suitable vehicle, e.g., sterile pyrogen-free water, before use.

The antibodies or fragments thereof described herein, including pharmaceutically acceptable salts and/or solvates thereof, are suitably used on their own but will generally be administered in the form of a pharmaceutical composition in which the one or more antibodies or fragments thereof described herein (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient, and from about 1 wt % to about 99.95 wt % or about 30 wt % to about 99.90 wt % of a pharmaceutically acceptable carrier, all percentages by weight being based on the total composition.

### IV. Kits

A further aspect relates to a kit comprising i) an antibody and/or binding fragment thereof described herein, ii) a nucleic acid of said antibody or a part thereof described herein, iii) composition comprising an antibody or fragment thereof described herein or iv) dosage form comprising an antibody or fragment thereof described herein, comprised in a vial such as a sterile vial or other housing and optionally a reference agent and/or instructions for use thereof.

In an embodiment, the kit is for diagnosing or monitoring HIT. In an embodiment, the kit further comprises one or more of a collection vial, standard buffer, PF4, PF4/heparin, and/or one or more detection reagents for detecting patient antibodies. In an embodiment, the kit further comprises one or more of a pre-coated PF4/PVS or PF4/heparin microwell strips, one or more buffers, including, for example, a standard wash buffer (e.g. PBS+detergent+blocking agent) and specimen dilutant (e.g. PBS+blocking agent, optionally including a visual dye), positive and negative controls, detection reagents (such as substrate (e.g. p-nitrophenylphosphate) and substrate buffer (DEA buffer)), and/or plastic plate sealers.

In another embodiment, the kit is for treating or preventing HIT. In an embodiment, the kit further comprises one or more of a sterile buffer for reconstitution and/or a syringe or other device for administration.

### V. Methods and Uses

As shown in the Examples, the antibodies and fragments thereof described herein can be use to distinguish between pathogenic (platelet-activating) and non-pathogenic (non-platelet activating) anti-PF4/heparin antibodies in patient samples. Specifically, the antibodies and fragments thereof described herein can distinguish between true positive “HIT positive” (EIA+/SRA+) and false positive “HIT negative” (EIA+/SRA−) patient samples. Accordingly, the antibodies and fragments thereof described herein can be used in diagnostic assays to rapidly identify patients with pathogenic anti-PF4/heparin antibodies and/or who have heparin-induced thrombocytopenia (HIT). Accordingly, provided herein are methods for identifying patients with pathogenic anti-PF4/heparin antibodies. Also provided herein are methods for diagnosing HIT. In an embodiment, the method for identifying patients with pathogenic anti-PF4/heparin antibodies or diagnosing HIT comprises a competitive binding assay, optionally an enzyme immunoassay (EIA). Antibodies suitable for use in the diagnostic assays and methods described herein typically a) lack an Fc domain, b) are a different class of antibody from the pathogenic antibodies, and/or c) are from a different species than the patient. For example, an scFv, a human IgM, or a mouse IgG comprising the CDRs and/or VL and VH domains described herein could be used in identifying human patients with pathogenic anti-PF4/heparin antibodies.

Any suitable competitive binding assay may be used, for example as described herein and in the Examples. In an embodiment, the method comprises obtaining a biological sample comprising patient antibodies to PF4/heparin from a patient, contacting the sample with i) PF4/heparin in the presence of an antibody or fragment thereof described herein, or a wildtype KKO antibody fragment, and ii) PF4/heparin in the absence of an antibody or fragment thereof described herein, or a wildtype KKO antibody fragment, under conditions permissive for forming PF4/heparin:patient antibody complexes; detecting the presence of any PF4/heparin:patient antibody complexes in i) and ii), wherein the detecting does not detect the antibody or antibody fragment described herein or the wildtype KKO antibody fragment; and determining the relative amount of PF4/heparin:patient antibody complexes in i) and ii) thereby determining if PF4/heparin:patient antibody binding is inhibited; wherein the patient is identified as having pathogenic anti-PF4/heparin antibodies and/or diagnosed with HIT if PF4/heparin:patient antibody binding is inhibited. In some embodiments, in i), the PF4/heparin is contacted with antibody or fragment thereof described herein or wildtype KKO fragment, prior to contacting with the sample.

As used herein, “wildtype KKO antibody fragment” refers to a KKO-derived antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23. In some embodiments, the wildtype KKO antibody fragment comprises a VL domain having an amino acid sequence of SEQ ID NO:9, and/or or a VH domain having an amino acid sequence of SEQ ID NO: 10, optionally the wildtype KKO antibody fragment comprises an amino acid sequence of SEQ ID NO: 3.

Suitable biological samples include, without limitation a liquid biopsy such as a blood sample, serum sample, or plasma sample. Accordingly, in an embodiment, the biological sample is a blood sample, serum sample, or plasma sample.

In an embodiment, the sample is obtained from a human patient. In an embodiment, the patient has been administered heparin, or is suspected of being at risk of developing HIT.

Any suitable method can be used to determine the presence of PF4/heparin:patient antibody complexes, for example by using an enzyme-linked or labeled antibody to specifically detect the presence of bound patient antibodies. For example, alkaline phosphatase-conjugated anti-human IgG and a suitable substrate e.g. p-nitrophenylphosphate (PNPP) can be used to detect anti-PF4/heparin antibodies from human patients by measuring absorbance at 405 nm (OD405 nm). Other enzyme conjugates (e.g. horseradish peroxidase) may also be used in combination with a suitable substrate. Conjugates comprising fluorophores or other dyes or labels may also be used.

The inhibition of binding may be determined using any suitable method, for example as a percent inhibition. By way of example, where detection of binding is determined using alkaline phosphatase and PNPP, percent inhibition can be calculated using the formula:

$$Percentage of inhibition ( \% ) = [ \frac{( OD 405 {nm}_{sera} - OD 405 {nm}_{KKO scFv + sera} )}{OD 405 {nm}_{sera}} ] \times 100$$

The above formula can be readily adapted for calculating the percent inhibition when using other enzyme/substrate combinations, fluorophores, etc. for detection. In an embodiment, the patient is identified as having pathogentic anti-PF4/heparin antibodies and/or diagnosed with HIT if the percent inhibition is greater than the positivity thresholds pre-determined for this assay using each individual scFv construct. For example, the pre-determined thresholds for wildtype, mutant B, and mutant F can be a percentage of inhibition greater or equal to (≥) 42.0%, ≥39.8%, and ≥54.6%, respectively, are determined to be positive for pathogenic HIT antibodies.

The antibodies and fragments thereof described herein are shown to inhibit platelet activation in a modified serotonin-release assay (SRA). Accordingly, provided herein are methods for inhibiting platelet activation in response to heparin in a patient in need thereof, the method comprising administering an effective amount of an antibody or fragment thereof described herein, or a wildtype KKO antibody fragment, to the patient. Also provided herein are a use of an antibody or fragment thereof described herein, or a wildtype KKO antibody fragment, for inhibiting platelet activation in a patient in need thereof. A further aspect includes the use of an antibody or fragment thereof described herein, or a wildtype KKO antibody fragment, in the manufacture of a medicament for inhibiting platelet activation. Another aspect includes an antibody or fragment thereof described herein, or a wildtype KKO antibody fragment, for use in inhibiting platelet activation. In these aspects, the antibody fragment does not comprise an Fc domain and/or does not interact with or form a complex with platelet Fc receptors.

Also provided herein are methods for treating or preventing HIT in a patient, the method comprising administering an effective amount of antibody or fragment thereof described herein, or a wildtype KKO antibody fragment, to the patient. Further provided herein is the use of an antibody or fragment thereof described herein, or a wildtype KKO antibody fragment, for treating or preventing HIT. Also provided herein is the use of an antibody or fragment thereof described herein, or a wildtype KKO antibody fragment, in the manufacture of a medicament for treating or preventing HIT. A further aspect includes an antibody or fragment thereof described herein, or a wildtype KKO antibody fragment, for use in treating or preventing HIT. In these aspects, the antibody fragment does not comprise an Fc domain and/or does not interact with or form a complex with platelet Fc receptors.

Optionally, the patient is identified as having pathogenic anti-PF4/heparin antibodies and/or diagnosed with HIT, for example using the methods described herein.

The term “patient” as used herein includes all members of the animal kingdom including mammals, and suitably refers to humans.

The term “patient in need thereof” refers to a patient that could benefit from the method(s) or treatment(s) described herein, and optionally refers to a patient identified as having pathogenic anti-PF4/heparin antibodies and/or diagnosed with HIT, and/or a heparin-treated patient.

The term “treating” or “treatment” as used herein and as is well understood in the art, means an approach for obtaining beneficial or desired results, including clinical results. Beneficial or desired clinical results include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. Treatment methods comprise administering to a patient a therapeutically effective amount of one or more antibodies or fragments thereof described herein and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition or manifesting a symptom associated with a disease, disorder or condition, for example HIT. For example, a patient identified as being at risk of developing HIT can be treated with one or more antibodies or fragments thereof described herein to prevent HIT from developing, or to reduce the severity or extent of HIT compared to expected severity or extent if not receiving preventative or prophylactic treatment. Prevention methods comprise administering to a patient a therapeutically effective amount of one or more antibodies or fragments thereof described herein and optionally consist of a single administration, or alternatively comprise a series of administrations.

The term “disease, disorder or condition” as used herein refers to a disease, disorder or condition treatable using the antibodies or fragments thereof, for example by blocking pathogenic anti-PF4/heparin antibody binding to PF4/heparin and/or preventing platelet activation by pathogenic anti-PF4/heparin antibodies.

The term “administered” or “administering” as used herein means administration of a therapeutically effective amount of a compound or composition of the disclosure to a patient. The antibodies or fragments thereof described herein may be administered using a variety of routes of administration. For example, the antibodies or fragments thereof described herein may be administered by parenteral administration. Optionally the antibodies or fragments thereof described herein may be administered by intravenous, intra-arterial, intraperitoneal, subcutaneous, or intramuscular administration. Optionally, the administration may be by continuous infusion over a selected period of time

The term “coadministration” or “combination therapy” shall mean that at least two compounds or compositions are administered to the patient at the same time, such that effective amounts or concentrations of each of the two or more compounds may be found in the patient at a given point in time. Although compounds according to the present disclosure may be co-administered to a patient at the same time, the term embraces both administration of two or more agents at the same time or at different times, provided that effective concentrations of all coadministered compounds or compositions are found in the patient at a given time.

As used herein, the phrase “effective amount” or “therapeutically effective amount” means an amount effective, at dosages and for periods of time necessary to achieve the desired result. For example in the context of treating or preventing HIT, an effective amount is an amount that for example prevents the occurrence of HIT, or reduces the severity or extent of HIT compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the administration schedule, the identity of the patient being treated, and the like, but can nevertheless be routinely determined by one skilled in the art.

Suitable administration schedules may include, without limitation, at least once a week, from about one time per two weeks, three weeks or one month, about one time per week to about once daily, 2, 3, 4, 5 or 6 times daily. The length of the treatment period may depend on a variety of factors, such as the severity of the disease, disorder or condition, the age of the patient, the concentration and/or the activity of the antibody or fragment thereof described herein and/or a combination thereof. It will also be appreciated that the effective dosage of the antibody or fragment thereof described herein used for the treatment may increase or decrease over the course of a particular treatment regime. Changes in dosage may result and become apparent by standard diagnostic assays known in the art. In some instances, chronic administration is required. For example, the antibody or fragment thereof described herein is administered to the patient in an amount and for duration sufficient to treat the patient.

The antibody or fragment thereof described herein may be either used alone or in combination with other known agents useful for treating diseases, disorders or conditions. When used in combination with other agents useful in treating such diseases, disorders or conditions, the antibody or fragment thereof described herein may be administered contemporaneously with those agents. As used herein, “contemporaneous administration” of two substances to a patient means providing each of the two substances so that they are both active in the individual at the same time. The exact details of the administration will depend on the pharmacokinetics of the two substances in the presence of each other, and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. In other embodiments, the combination of agents is administered to a patient in a non-contemporaneous fashion. In an embodiment, the antibody or fragment thereof, composition, etc. described herein is administered with another therapeutic agent simultaneously or sequentially in separate unit dosage forms or together in a single unit dosage form. Accordingly, the present description provides a single unit dosage form comprising the antibody or fragment thereof, composition, etc. described herein, an additional therapeutic agent, and a pharmaceutically acceptable carrier.

The dosage of the antibody or fragment thereof described herein varies depending on many factors such as the pharmacodynamic properties of the antibody or fragment, the mode of administration, the age, health and weight of the recipient, the nature and extent of the symptoms, the frequency of the treatment and the type of concurrent treatment, if any, and the clearance rate of the compound/cell in the patient to be treated. One of skill in the art can determine the appropriate dosage based on the above factors. In some embodiments, the antibody or fragment thereof described herein is administered initially in a suitable dosage that is adjusted as required, depending on the clinical response. Dosages will generally be selected to maintain sufficient levels of the antibody or fragment thereof, composition, etc. described herein.

### EXAMPLES

The following non-limiting examples are illustrative of the present application:

### Example 1. Materials and Methods

Phage Display Library Construction. The M13 filamentous phage display vector, pADL-22c, was purchased from Antibody Design Laboratories Inc. (San Diego, CA). A library of mutant scFv sequences was first constructed for expression in the pADL-22c phage display system. In PCR reactions, primers SI and AS1 (Table 1) were used to amplify the variable heavy and light chain regions of KKO using the BioRad T100 Thermal Cycler (BioRad, Hercules, CA). Mutagenic PCR was performed using Mutazyme II Polymerase (Agilent Technologies, Inc., Santa Clara, CA), according to manufacturer's instructions. scFv PCR products were digested with Sfi1 and cloned into the overlapping BglI restriction sites of pADL-22c using T4 DNA Ligase (New England Biolabs Ltd., Ipswich, MA). Ligated products were electroporated into TG1 Electrocompetent E. coli cells (Lucigen, Middleton, WI) using the BioRad Gene Pulser (BioRad). Library depth was quantified by determining the number of colony forming units per mL (CFU) on LB agar plates supplemented with 100 μg/mL ampicillin. Colonies were then randomly selected to confirm scFv insertion by sanger sequencing.

TABLE 1
Primers for scFv amplification from pADL-22c phagemid.
Underlined amino acids represent the BglI/SfiI overlapping
restriction enzyme recognition sites.

| Primer” | Sequence |
|---|---|
| S1...Sense Primer | 5′ |
| (SEQ ID NO: 1) | GTTATTACTCGCGGCCCAGCCGGCCATGGCTGATAT |
| | CCAGATGATCCAGAGCC 3′ |
| AS1...Anti-Sense | 5′ |
| Primer | CCATGATGGTGGTGATGGTGTTGGCCTCCCGGGCCA |
| (SEQ ID NO: 2) | CTAGTGGCAGACACGGTAACCAGG 3′ |

Phage Library Preparation and Purification. Phage library preparation and purification were performed using a polyethylene glycol (PEG)/NaCl precipitation method as described by Barbas et al. (2001)³⁶ and Kretz (2017).³⁷ A culture of transformed TG1 E. coli (Lucigen) was grown at 37° C. while shaking at 250 rpm to the mid-exponential phase (OD₆₀₀=0.5-0.6 nm) in LB media containing 100 μg/mL ampicillin. The helper phage M13K07 was then added and incubated with the E. coli culture for 1 h at 37° C. while shaking at 250 rpm. Culture media was then centrifuged (6000×g, 15 min) to pellet bacteria. The bacterial pellet was then resuspended in 2×YT media containing 100 μg/mL ampicillin and 50 μg/mL kanamycin and incubated overnight at 30° C. while shaking at 250 rpm. The following day, culture media was centrifuged (6000×g, 10 min) to remove cell debris and phages were precipitated from the supernatant with 0.15× vol of PEG/NaCl (4% w/v PEG-8000+3% w/v NaCl) at 4° C. overnight. The phages were pelleted by centrifugation (6500×g, 60 min, 4° C.) and re-suspended in Tris buffered saline (TBS) (50 mM Tris-HCl, pH 7.5, 150 mM NaCl). The supernatant was cleared of debris by centrifugation (10 000×g, 10 min) followed by an additional round of precipitation before storing in TBS with 0.1% v/w Tween-20 (TBS-T) at 4° C. Phage preparations were titered by first growing a fresh culture of K91 E. coli in LB media containing 50 μg/mL kanamycin followed by infection with eluted phages for 1 h at 37° C. while shaking.

Phage titers were calculated according to the following formula:

$$\frac{\# of colony forming units ( CFU )}{100 μL \times dilution factor ( {d}_{f} )} = {C}_{f} ( phage / μL )$$

Fluid-Phase Phage Bio-Panning Using Streptavidin Coated Magnetic Beads. Streptavidin-coated magnetic beads were pre-washed with 0.1% v/w Tween-20 in Tris buffered saline (TBS-T, 50 mM Tris-HCl, pH 7.5, 150 mM NaCl). To immobilize the target antigen, biotinylated-heparin (0.5 U/mL) and PF4 (30 μg/mL) in TBS-T supplemented with 2% BSA were incubated with 25 μL streptavidin-coated magnetic beads (Dynabeads™ M-450 Streptavidin, ThermoFisher, Hercules, CA) for 1 h at room temperature while rotating. All wash steps were performed by adding 1 mL of TBS-T and incubating while rotating for 3 minutes before magnetically separating and discarding the supernatant. After washing 3× with TBS-T, beads were incubated with phage in TBS-T+2% BSA for 2 h at room temperature while rotating. Beads were then washed 5×3 min with TBS-T and magnetically separated. After the final wash, phages were eluted in 0.1 M Glycine-HCl (pH 2) and incubated for 10 min at room temperature while rotating. The eluted phage solution was neutralized by adding Tris-HCl buffer (pH 9). A fresh culture of K12 ER2738 F'pilus positive E. coli (New England Biolabs Ltd.) was grown simultaneously (OD₆₀₀ₙₘ=0.5) in LB media containing 10 μg/mL tetracycline and infected with eluted phages for 1 h at 37° C. while shaking. An aliquot of the infected culture was then incubated overnight on LB agar+2% glucose (w/v)+100 μg/mL ampicillin plates at 37° C. and used for sequence analysis by PCR. The remaining culture was pelleted and resuspended into fresh LB media+2% glucose (w/v)+100 μg/mL ampicillin and grown up overnight to amplify the phage. The following day, a new phage library preparation was performed as described in section 3.2. Five rounds of selection were performed before selecting lead candidate scFv mutants based on sequence analysis.

Construction and Bacterial Expression of Wildtype and Mutant scFv. The vector pADL-22c expressing the heavy and light chain variable regions (scFv) of KKO was used for protein expression in bacterial cells. scFv was transformed into chemically competent E. coli One Shot® BL21 (DE3) cells (ThermoFisher) before induction. To overexpress scFv, bacterial cultures were grown at 37° C. to the mid-exponential phase (OD₆₀₀ₙₘ=0.7-0.8), before adding 0.5 mM isopropyl β-D-1-thiogalactopyranoside (IPTG) to induce protein production while shaking at 225 rpm and 37° C. for 3 h. Following induction, culture media was centrifuged at 5000 rpm for 15 min to harvest cells. Protein expression was then assessed by examining the pellet of each E. coli strain on a 4-20% SDS polyacrylamide gradient gel (BioRad). Bacterial pellets collected from 1 L of culture media were then re-suspended in 50 mL lysis buffer: 50 mM sodium phosphate, pH 8, 300 mM NaCl, 20 mM imidazole, 5% (v/v) glycerol, 1% (v/v) Triton-X 100 (ThermoFisher), and 0.5% (v/v) DOC (SigmaAldrich, Oakville, ON) with protease inhibitors (Roche complete EDTA free protease inhibitor tablets, Roche, Laval, QC). Cell lysis was carried out by sonication in 5 s pulses at 60% amplitude with 5 s cooling cycles on ice for a total of 4 min. The supernatant was then centrifuged at 12,000×g for 30 min and collected for purification by Ni²⁺ affinity chromatography.

Ni-NTA Affinity Purification of scFv. Empty gravity flow chromatography columns (BioRad) loaded with 1 mL HisPur™ Ni-NTA Resin (ThermoFisher) were used for scFv purification. The supernatant collected following bacterial lysis was added to the Ni-NTA column followed by 2×50 mL washes with buffer (50 mM NaPO₄, pH 8, 300 mM NaCl, and 20 mM imidazole). Histidine-tagged scFv was then eluted in a buffer solution containing 50 mM NaPO₄, pH 8, 300 mM NaCl, and 500 mM imidazole. Eluted fractions with the highest absorbance at 280 nm were then pooled and concentrated using Amicon® Ultra-4 10K centrifugal filter units (MilliporeSigma, Burlington, MA). Purification was confirmed by examining a sample of each eluted fraction on a denaturing 4-20% SDS polyacrylamide gradient gel (BioRad) and visualized using Coomassie SimplyBlue™ SafeStain (ThermoFisher).

Inhibitory PF4/heparin IgG-specific Streptavidin Enzyme Immunoassay (EIA). The binding activity of wildtype and mutant scFv antibodies to PF4/heparin complexes was measured using an inhibitory PF4/heparin IgG-specific EIA with a streptavidin-biotin capture system. Before performing this protocol, 96-well NUNC Maxisorp plates (ThermoFisher) were coated with 10 μg/mL streptavidin for 24 h. Plates were then blocked with 3% bovine serum albumin (BSA) in phosphate buffered saline (PBS) for 2 h at room temperature. Plates were then coated with 1 U/mL biotinylated-heparin for 1 h followed by 30 μg/mL PF4 for 1 h. Increasing concentrations of wildtype or mutant scFv were added and incubated for 1 hr at room temperature. Without removing wildtype or scFv, HIT patient sera in a 1:50 dilution or full-length KKO (2 μg/mL) was then incubated for 1 h at room temperature. After washing, alkaline phosphatase-conjugated anti-human or anti-mouse IgG (Jackson Immuno Research Laboratories, Inc., West Grove, PA) was added at a 1:4000 dilution and incubated for 1 h temperature. For detection, 1 mg/mL p-nitrophenylphosphate (PNPP, Sigma-Aldrich) substrate dissolved in 1 mol/L diethanolamine (DEA) buffer (pH 9.6) was added. The optical density (OD) was measured at 2 min intervals for 30 min at 405 nm (OD₄₀₅ₙₘ) using a BioTek 800 TS plate reader (BioTek Instruments Inc., Winooski, VT) to determine if anti-PF4/heparin antibody binding was inhibited. Results were reported as a decrease in OD (absorbance at 405 nm) from baseline levels.

The percentage of inhibition relative to anti-PF4/heparin antibody binding in the absence of scFv was also calculated using the following formula:

$$Percentage of inhibition ( \% ) = [ \frac{( OD 405 {nm}_{sera} - OD 405 {nm}_{KKO scFv + sera} )}{OD 405 {nm}_{sera}} ] \times 100$$

The concentration of scFv achieving 50% inhibition (IC50) of KKO binding was calculated using non-linear dose-response curve fitting analysis on GraphPad Prism version 9.1.2 (GraphPad Software, Inc).

Biolayer Interferometry (BLI) using Wildtype and Mutant scFv. PF4 was purified and biotinylated as previously described.^{38,39} Briefly, PF4 was incubated with 5× Heparin Sepharose 6 Fast Flow Chromatography Medium (GE Healthcare, Chicago, IL) for 1 h while shaking before adding 20 molar excess EZ-link-Sulfo-NHS-LC-Biotin (ThermoFisher). The biotin-PF4-heparin-sepharose mixture was incubated for 1 h with shaking to allow for the biotinylating reaction to occur. Biotinylated-PF4 was eluted from the heparin sepharose at high sodium concentration (PBS+2M NaCl) and the absorbance at 280 nm was measured on a spectrophotometer (Eppendorf, Hamburg, Germany) to determine the concentration. Biotinylation of PF4 was then confirmed in a streptavidin anti-PF4/heparin EIA using the murine monoclonal antibody KKO.

The Octet-QK Red 96 (FortéBio, Menlo Park, CA) instrumentation was used to conduct all BLI experiments. A 96-well microtiter plate (black, flat-bottom) was used to hold 200 μL per well of either sample or buffer. Pre-coated streptavidin biosensor tips (FortéBio) were hydrated in phosphate buffered saline (PBS) containing 1% bovine serum albumin (BSA, Sigma-Aldrich) before performing all kinetic assays. Each step was performed at an operating temperature of 23° C. with agitation at 1000 rpm. Streptavidin tips were first equilibrated in PBS+1% BSA to establish a baseline. Biosensors were then dipped into wells containing 7.5 μg/mL biotinylated-PF4 (7.5 μg/mL in PBS+1% BSA) alone or complexed with 0.125 U/mL unfractionated heparin for 15 minutes for antigen immobilization. The PF4-coated tips were then dipped into PBS+1% BSA for 13 minutes to re-establish a baseline. Tips were then incubated in an analyte solution containing half-fold dilutions of wildtype scFv (4.0, 2.0, 1.0, 0.5, 0.25 μg/mL in PBS+1% BSA) for 10 minutes to measure association. For mutant scFv association, tips were incubated in an analyte solution containing 200 μL of each mutant and wildtype scFv at a final concentration of 1 μg/mL in PBS+1% BSA. Antibody dissociation steps were performed by dipping sensors in wells containing PBS+1% BSA for 56 minutes. The following negative controls included in these kinetic experiments were also measured: 200 μL of buffer to replace both the antigen and scFv and 200 μL buffer replacing scFv only. Each experiment was performed with PF4 or with PF4/heparin as the immobilized antigen.

BLI Data Acquisition and Kinetic Analysis. Data acquisition was performed using Octet® User Software version 3.1 and analyzed using the 1:1 homogenous ligand binding model. Results were processed by subtracting reference from control values and aligned to fit curves correctly according to the baseline. Average rate constants (±standard deviation) for association (kₒₙ), dissociation (k_{off}), and affinity (K_{D}) along with error and R² values were calculated automatically by the supplier's software. The binding profile of each analyte was also described in terms of an average wavelength shift (nm; response), representing the change in molecules bound to the biosensor surface for the duration of the association step.

Epitope Mapping of KKO, and Wildtype and Mutant scFv Binding to PF4. Epitope mapping was performed as described in a modified streptavidin PF4/heparin IgG-specific EIA using a library of 70 PF4 mutants.^{19,40} Previously, the DNA coding sequence of human PF4 was expressed in the pET22b expression vector at the NdeI and HindIII restriction enzyme sites (GenScript, Piscatawa, NJ). Mutants of PF4 were designed using alanine-scanning mutagenesis, where each residue of PF4 was mutated to alanine or valine in the case of an alanine residue.¹⁹ The purification of mutant PF4 was also performed as previously described.^{19,38} Mutant PF4 was transformed and over-expressed in ArcticExpress (DE3) E. coli cells (Agilent Technologies) at 37° C. before inducing at 37° C. for 3h in the presence of 0.5 mM IPTG. Cells containing mutant PF4 were lysed by sonication in a buffer (pH 7.2) containing 20 mM Na₂PO₄, 400 mM NaCl, 1.4 mM β-Me, 5% (v/v) glycerol, 1% (v/v) Triton X-100 (ThermoFisher), 0.5% (w/v) DOC (MilliporeSigma), 2 mM MgCl₂, 10 μg ml⁻¹ DNaseI (MilliporeSigma), and EDTA-free protease inhibitor cocktail (Roche). Lysis supernatant was then centrifuged for 40 min at 40,000×g and purified in a two-step process first loading the supernatant on a HiTrap Q HP column (Cytiva Life Sciences, Marlborough, MA) followed by purification on a HiTrap Heparin HP column (Cytiva Life Sciences). Each PF4 mutant was eluted from the column on a linear gradient of 0.5 to 2M NaCl and fractions containing pure protein were concentrated and stored in PBS.

The binding of scFv to mutant or wildtype PF4 was then assessed in a modified streptavidin PF4/heparin IgG-specific EIA as performed by Huynh et al. (2019, 2021).^{19,40} NUNC Maxisorp 384-well microtiter plates (ThermoFisher) were coated with 10 μg/mL streptavidin for 24 h. Plates were then blocked with 3% BSA in PBS for 2 h at room temperature and coated with 1 U/mL biotinylated-heparin for 1 h. Wild-type or mutant PF4 using a concentration of 5 μg/mL were then incubated for 1 h at room temperature followed by diluted scFv (wildtype and mutant) at a concentration of 5 μg/mL. To detect scFv binding, an anti-6× histidine-tag antibody (ThermoFisher) at a 1:10,000 dilution was added for 1 h at room temperature. After washing, alkaline phosphatase-conjugated anti-mouse IgG (Jackson Immuno Research Laboratories, Inc.) was incubated on plates for 1 h at a 1:4000 dilution. Finally, 1 mg/mL PNPP (Sigma-Aldrich) substrate dissolved in 1 mol/L DEA buffer (pH 9.6) was added. The OD was measured at 2 min intervals for 30 min at 405 nm (OD₄₀₅ₙₘ) and 490 nm (OD₄₉₀ₙₘ) using a BioTek 800 TS plate reader (BioTek Instruments Inc.) to determine if scFv binding was perturbed. An amino acid was defined as essential to the scFv epitope if it resulted in a greater than (≥) 50% reduction in binding when mutated to alanine or valine compared to the wildtype residue. The percentage of binding loss relative to wildtype PF4 was calculated using the following formula:

$$\% binding relative to WT - PF 4 = \frac{{( OD 405 {nm}_{mutant PF 4} / OD 405 {nm}_{WT PF 4} )}_{Test ab}}{{( OD 405 {nm}_{mutant PF 4} / OD 405 {nm}_{WT PF 4} )}_{Polyclonal HIT - ab}} \times 100$$

Serotonin-Release Assay (SRA) using scFv. Previously tested EIA-positive/SRA-positive HIT patient samples (n=5) were tested in a modified SRA to determine if scFv can inhibit platelet activation. Platelets labelled with ¹⁴C-serotonin were pre-incubated with 50 μg/mL and 75 μg/mL of wildtype or mutant scFv constructs for 15 min. Platelets and scFv were then incubated with patient sera in the presence of therapeutic heparin doses (0.2 U/mL) as previously described.⁴¹ Plates were incubated for 1 hr with shaking, followed by the addition of PBS-EDTA before centrifuging. To determine ¹⁴C-serotonin-release, an aliquot of supernatant was removed from each well and measured using a scintillation counter (Packard Topcount, Meriden, CT, USA). The ability of each scFv construct to inhibit immune complex formation resulting in a moderate (≥20%) or strong (≥50%) decrease in ¹⁴C-serotonin release was determined relative to control wells containing patient sera in the absence of scFv.

GraphPad Prism version 9.1.2 for Mac OS software (GraphPad Software, Inc) was used to create all graphs and perform statistical analysis. Differences between data sets were tested for statistical significance using an unpaired, two-tailed, t-test using Microsoft Excel for Mac version 15.37 where p<0.01 was considered statistically significant.

### Example 2. Results

Isolation of scFv clones from a phage display library: A large phage library was screened for scFv mutants specific for complexes of PF4/heparin. Five rounds of panning were performed to enrich scFv mutants and eliminate non-specific, weak, and/or unbound phage through various stringent washing steps (Table 2). At each cycle of bio-panning, input and output phage titers were monitored followed by Sanger sequencing (Table 2).

TABLE 2
Phage library selection and enrichment process. Each round of bio-panning
was performed using PF4 (30 μg/mL) and heparin (0.5 U/mL) complexes as the target
antigen. Stringent washing steps were performed to remove unbound, non-specific, and
weakly bound phage displaying scFv mutants. The number of input and out phage titers
(phage/μL) were tracked throughout each cycle to monitor enrichment.

| Panning | | Tween- | No. of | Input Phage | Output Phage |
|---|---|---|---|---|---|
| Round | Antigen | 20 (%) | Washings | (phage/uL) | (phage/uL) |
| 1 | PF4 (30 μg/mL) | 0.1 | 3 × 5 | 6.8 × 10⁹ | 6.0 × 10⁵ |
| | Heparin (0.5 U/mL) | | min | | |
| 2 | PF4 (30 μg/mL) | 0.1 | 3 × 5 | 2.0 × 10⁷ | 5.0E × 10⁵ |
| | Heparin (0.5 U/mL) | | min | | |
| 3 | PF4 (30 μg/mL) | 0.1 | 3 × 5 | 3.5 × 10⁸ | 4.6 × 10⁶ |
| | Heparin (0.5 U/mL) | | min | | |
| 4 | PF4 (30 μg/mL) | 0.1 | 3 × 5 | 2.0 × 10⁷ | 9.6 × 10⁵ |
| | Heparin (0.5 U/mL) | | min | | |
| 5 | PF4 (30 μg/mL) | 0.1 | 3 × 5 | 2.4 × 10¹⁰ | 3.7 × 10⁶ |
| | Heparin (0.5 U/mL) | | min | | |

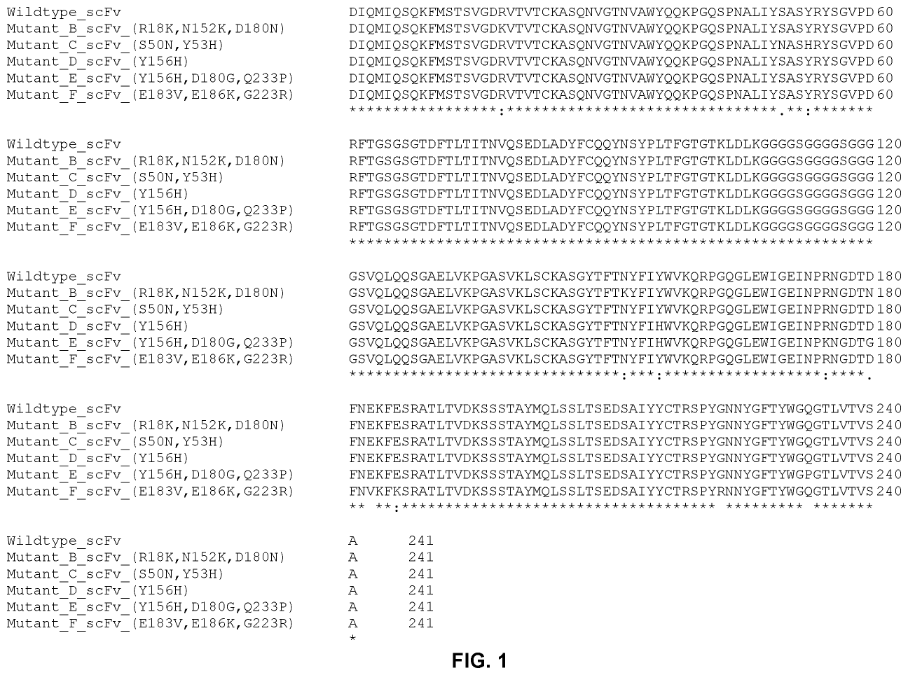

Sequence analysis was performed on 40 randomly chosen colonies at each round, identifying five frequently occurring scFv mutants with unique sequences (Table 3). The lead candidate mutants observed include: scFv^{(R18K, N152K, D180N)}, scFv^{(S50N, Y53H)}, scFv^{(Y156H)}, scFv^{(Y156H, D180G, Q233P)}, and scFv^{(E183V, E186K, G223R)}, henceforth referred to as mutant B, C, D, E, and F, respectively (FIG. 1). After five rounds, the frequency of mutant B was 7.5%, mutant C was 7.5%, mutant D was 15%, mutant E was 12.5%, and mutant F was 4% (Table 3). Additionally, an identical mutation (Y156H) was observed in both mutant D and mutant E along with another overlapping mutation at position D180 between mutant B (D180N) and mutant E (D180G). This observation suggests these two positions (Y156 and D180) may be important sites within the scFv paratope.

TABLE 3
Frequency of identical mutant sequences. Lead candidate scFv
mutants were selected based on enrichment following
five rounds of bio-panning.

| | | Frequency | Frequency |
|---|---|---|---|
| Mutant ID | Grouped Mutations | (/Total Colonies) | (%) |
| B | R18K, N152K, D180N | 3/40 | 7.5 |
| C | S50N, Y53H | 3/40 | 7.5 |
| D | Y156H | 6/40 | 15.0 |
| E | Y156H, D180G, Q233P | 5/40 | 12.5 |
| F | E183V, E186K, G223R | 2/48 | 4.0 |

A number of key mutations also appeared within the complementary determining regions (CDR), which are six hypervariable loops located between both the VH and VL domains that form the primary antigen binding site.⁴² The two previously highlighted mutated residues of interest (Y156 and D180) were both located within the VH CDR, specifically CDR-H1 and CDR-H2, respectively. In fact, the four strongest affinity scFv variants (mutant B, D, E, and F) had at least two or all mutations identified on the CDR loops found within the VH domain, which are primarily involved in antigen binding. The SEQ ID NOs: for each of the VH, VL, and CDR domains are listed in Table 4.

TABLE 4
SEQ ID NOs: for variable light and heavy domains and CDR
domains for wild-type scFv and various mutant scFvs

| | VL | VH | CDRL1 | CDRL2 | CDRL3 | CDRH1 | CDRH2 | CDRH3 |
|---|---|---|---|---|---|---|---|---|
| WT | 9 | 10 | 18 | 19 | 20 | 21 | 22 | 23 |
| Mut B | 11 | 12 | 18 | 19 | 20 | 24 | 25 | 23 |
| Mut C | 13 | 10 | 18 | 26 | 20 | 21 | 22 | 23 |
| Mut D | 9 | 14 | 18 | 19 | 20 | 27 | 22 | 23 |
| Mut E | 9 | 15 | 18 | 19 | 20 | 27 | 28 | 23 |
| Mut F | 9 | 16 | 18 | 19 | 20 | 21 | 29 | 30 |

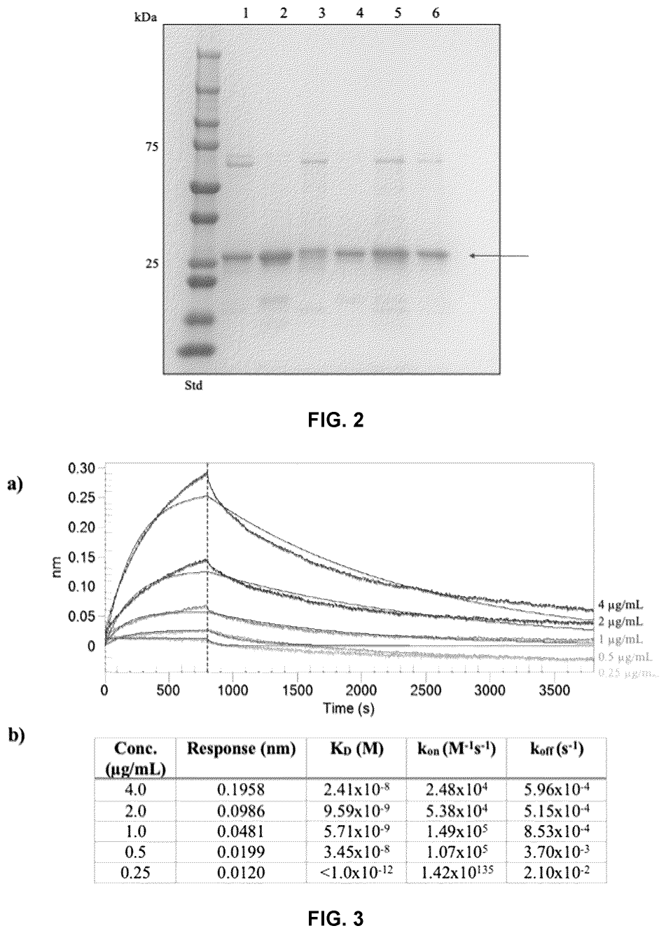

After identifying five novel scFv variants, each mutant and wildtype were expressed in BL21 E. coli and purified on a Ni-NTA column for further characterization (FIG. 2). After affinity purification, the protein yield for wildtype scFv was 2.16 mg, mutant B was 6.00 mg, mutant C was 1.15 mg, mutant D was 5.29 mg, mutant E was 2.33 mg, and mutant F was 1.42 mg. Concentrated fractions containing pure protein as determined by measuring absorbance at 280 nm were separated on a 4-20% SDS PAGE gel. scFv migrated as a single band with relative mobility of approximately 28 kDa, which was observed following Coomassie SimplyBlue™ SafeStain analysis (FIG. 2).

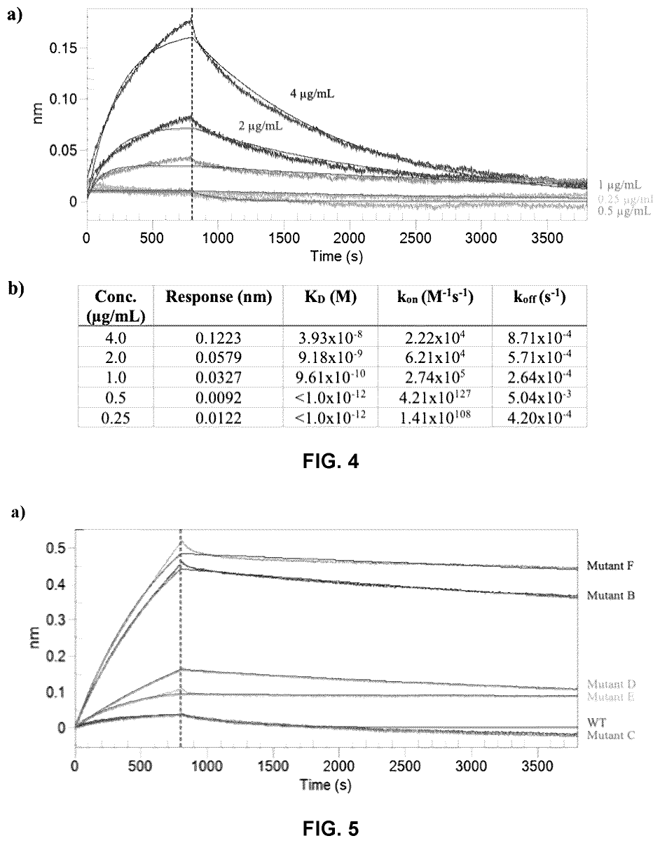

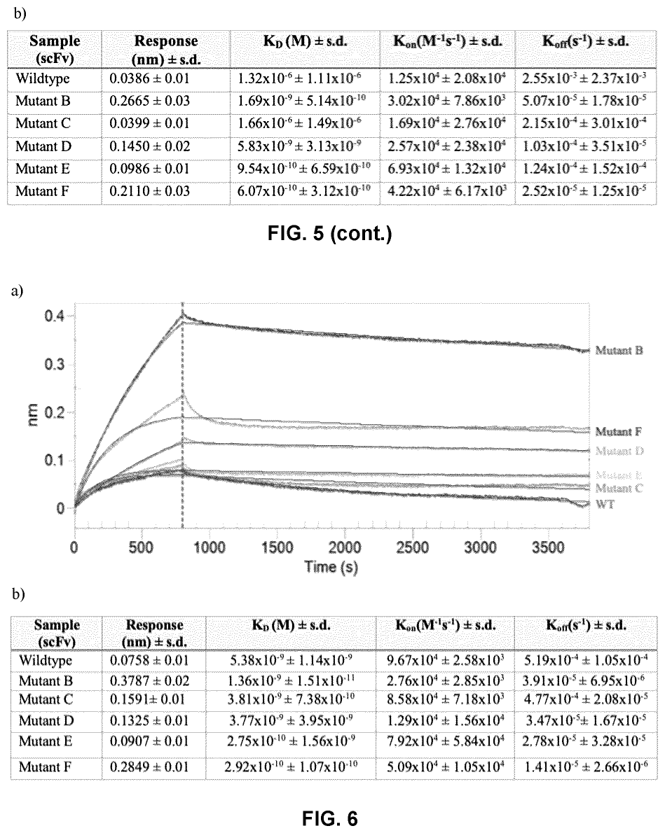

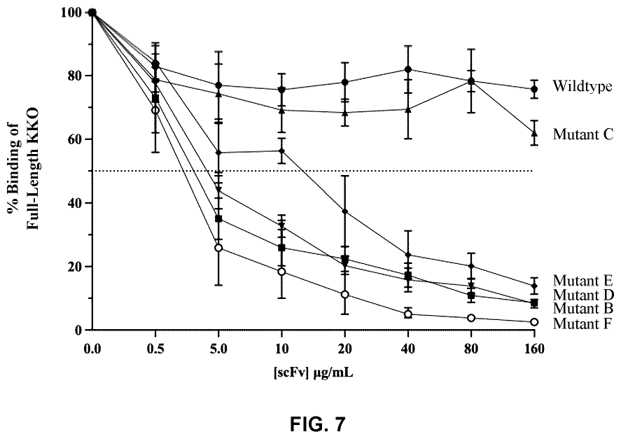

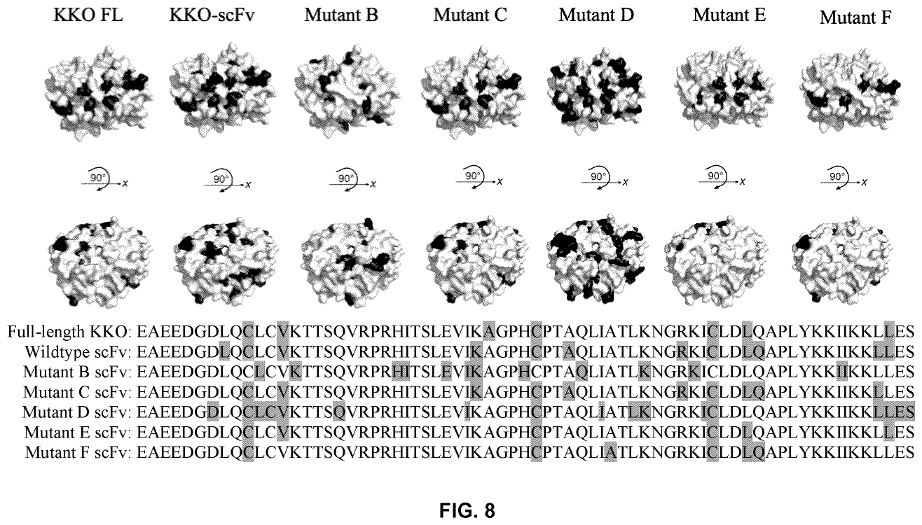

BLI experiments were performed to evaluate binding responses and affinities of wildtype and mutant scFv. Biotinylated PF4 alone or complexed with heparin was immobilized on streptavidin biosensors and used to capture wildtype scFv. Half-fold serial dilutions of wildtype scFv ranging from 0.25 μg/mL to 4 μg/mL were used to determine the optimal concentrations for future experiments PF4 (PF4 FIG. 3; PF4/heparin FIG. 4). An optimal concentration of 1 μg/mL was selected and BLI was repeated with immobilized biotinylated PF4 alone (FIG. 5) or complexed with heparin (FIG. 6) to compare average binding responses [wavelength shift (nm)±standard deviation (s.d.)] and kinetic rates [equilibrium constant (KD)±s.d.] of wildtype to each mutant scFv.

BLI analysis revealed that wildtype scFv had an average binding response of 0.0386±0.01 nm against PF4 and 0.0758±0.01 nm against PF4/heparin (FIG. 5b, 6b). Mutant C showed comparable binding responses to wildtype scFv for PF4 (0.0399±0.01 nm) but had 2-fold higher binding responses to PF4/heparin (0.1591±0.01 nm) (FIG. 5b, 6b). Mutant D demonstrated at least 3-fold higher binding responses than wildtype when tested against PF4 alone (0.1450±0.02 nm) and PF4/heparin (0.1325±0.01 nm) (FIG. 5b, 6b). Mutant E had a 2-fold higher binding response to PF4 (0.0986±0.01 nm) and a similar binding response when tested against PF4/heparin (0.0907 nm s.d.±0.01 nm) (FIG. 5b, 6b). The binding response of mutant B had a 7-fold increase against PF4 (0.2665±0.03 nm) and 4-fold increase (0.3787±0.01 nm) against PF4/heparin (FIG. 5b, 6b). Lastly, mutant F had a 5-fold increase binding response (0.2110±0.03 nm) against PF4 and 5-fold increase binding response (0.2849±0.01 nm) against PF4/heparin compared to wildtype (FIG. 5b, 6b).

The average K_{D} values for wildtype scFv were calculated using the kon and koff rates for PF4 (K_{D}=1.32×10⁻⁶±1.11×10⁻⁶ M) and PF4/heparin (K_{D}=5.38×10⁻⁹±1.14×10⁻⁹ M) (FIG. 5b, 6b). Mutant C had a comparable affinity as wildtype scFv for PF4 alone (1.66×10⁻⁶±1.49×10⁻⁶ M) and a 10-fold increased affinity for PF4/heparin (3.81×10⁻⁹±7.38×10⁻¹⁰ M) (FIG. 5b, 6b). Mutant D had a 1000-fold stronger affinity (5.83×10⁻⁹ M+3.13×10⁻⁹ M) and a 100-fold stronger affinity (3.77×10⁻⁹±3.95×10⁻⁹ M) compared to wildtype scFv, while mutant E had 10,000-fold (9.54×10⁻¹⁰±6.59×10⁻¹⁰ M) and 100-fold (2.75×10⁻¹⁰±1.56×10⁻⁹ M) stronger affinities when tested against PF4 and PF4/heparin, respectively (FIG. 5b, 6b). The K_{D} for Mutant B against PF4 alone increased by 1000-fold (1.69×10⁻⁹±5.14×10⁻¹⁰ M) and 100-fold (1.36×10⁻⁹±1.51×10⁻¹¹ M) against PF4/heparin (FIG. 5b, 6b). Mutant F also demonstrated improved affinities, with a K_{D} of 6.07×10⁻¹⁰±3.12×10⁻¹⁰ M) against PF4 and 2.92×10⁻¹⁰±1.07×10⁻¹⁰ M against PF4/heparin (FIG. 5b, 6b). The data presented here demonstrate at least four mutant variants of scFv out of the five isolated have stronger binding responses and increased affinity for PF4 alone, and all five mutant variants have stronger binding responses and increased affinity for PF4/heparin, suggesting these mutants have an improved kinetic performance compared to wildtype.

To further evaluate the binding of mutant and wildtype scFv, their ability to inhibit full-length KKO from binding PF4/heparin complexes was assessed in a modified streptavidin-capture EIA. Biotinylated heparin was immobilized with PF4 on streptavidin-coated plates and incubated with KKO in the presence of scFv at half-fold serial dilutions from 0 to 160 μg/mL. The concentration that achieves 50% inhibition (IC50) of KKO binding was then determined for wildtype and each mutant scFv variant, where lower IC50 values reflect mutants with superior inhibitory strength. Wildtype scFv and mutant C were unable to inhibit full-length KKO binding at these concentrations and IC50 values for these constructs were not calculated. Mutant B had an IC50 value of 57.3 nM (1.6 μg/mL), mutant D had an IC50 value of 111.1 nM (3.1 μg/mL), mutant E had an IC50 value of 292.8 nM (8.2 μg/mL), and mutant F had an IC50 value of 46.8 (1.3 μg/mL).

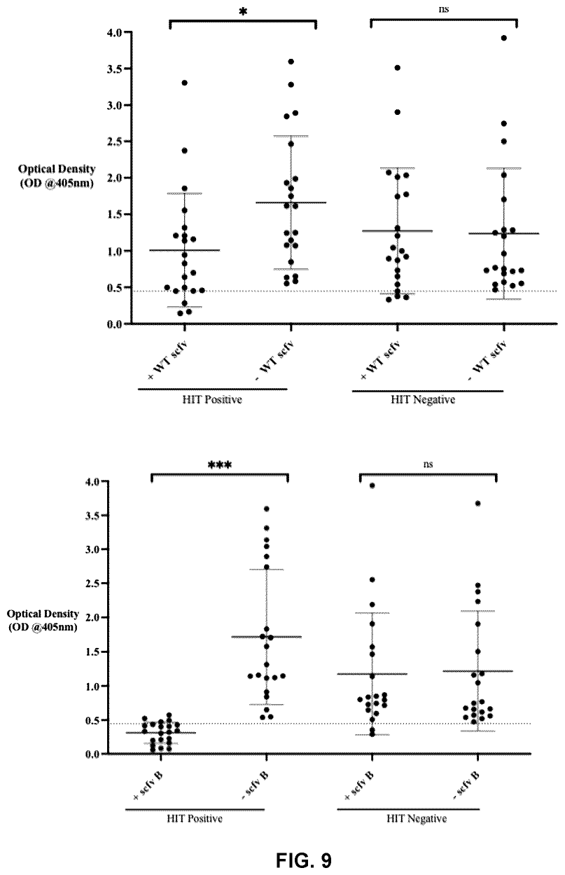

Wildtype scFv was unable to significantly prevent full-length KKO at any concentration, achieving a maximum inhibition of 24.2±4.9% (FIG. 7). Mutant B appeared much stronger than wildtype scFv, illustrated by the substantial improvement of KKO inhibition and effectiveness at low concentrations (IC50=1.6 μg/mL) (FIG. 7). Mutant C had a similar response against KKO to the wildtype, with weak inhibitory activity at all concentrations and a maximum of only 38.0±6.7% inhibition (FIG. 7). Mutant D and E also showed improved inhibitory activity against KKO at high and low concentrations compared to wildtype, with low IC50 values (3.1 μg/mL and 8.2 μg/mL, respectively) (FIG. 7). Mutant F demonstrated the strongest inhibitory activity against KKO at all concentrations with the lowest IC50 value (1.3 μg/mL). These results also suggest scFv mutant B, D, E, and F have improved binding affinities for PF4/heparin as indicated by their stronger inhibitory effect on full-length KKO compared to wildtype, supported by earlier BLI data.

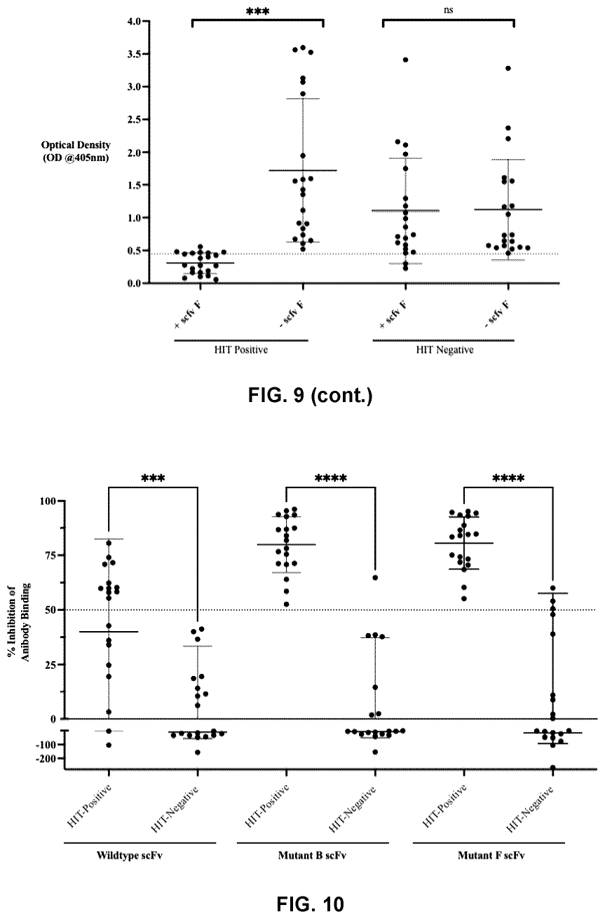

Epitope mapping was then done using a library of 70 PF4 mutants, previously generated by alanine-scanning mutagenesis, to identify the wildtype scFv binding site. Amino acids critical for scFv binding were identified as those causing a reduction in binding by 60% or greater when mutated to an alanine residue (or valine if the wildtype amino acid was alanine) compared to wildtype PF4. Epitope mapping identified thirteen key amino acids (L8, C10, V13, A32, K31, C36, A39, I41, C52, L55, Q56, L67, and L68) that compose the binding site for wildtype scFv. Comparing the binding site also revealed eight critical amino acids (C10, V13, A32, C36, C52, L55, Q56, and L68) overlap between the wildtype scFv and full-length KKO binding site on PF4^{19,43} (FIG. 8b). As predicted based on previous epitope mapping studies with KKO,¹⁹ these key amino acids were also localized to the same region when displayed on the PF4 tetramer (FIG. 8a).

To show any changes to the wildtype scFv binding site, epitope mapping of each mutant construct was then performed (FIG. 8). The epitope for mutant C was found to contain eight amino acids on PF4 (C10, V13, A32, C36, C52, L55, Q56, L67), seven of which correspond to amino acids on PF4 bound by both full-length KKO and wildtype scFv. Epitope mapping further identified 20 amino acids on PF4 that make up the mutant D binding site (N7, C10, L11, C12, V13, Q18, I30, C36, L45, K46, I51, C52, L55, Q56, A57, Q56, L67, L68, Q69, S70), of which eight amino acids were shared by both full-length KKO and wildtype scFv and overlapped with the same region on PF4 (FIG. 8). The mutant E scFv epitope was found to contain 6 amino acids on PF4 (C11, V14, C37, C53, L56, L69), of which all overlapped with the epitope for KKO and wildtype scFv (FIG. 8). Mutant F epitope mapping revealed 6 amino acids (C11, C37, A44, C53, L56, Q57) critical for binding to PF4 (FIG. 8). Of these 6 amino acids, 4 were shared with the KKO epitope and 5 were shared with the scFv epitope (FIG. 8b).

Epitope mapping of mutant B revealed this construct bound different amino acids on PF4 compared to wildtype scFv (FIG. 8). None of the 11 identified residues (L11, K14, H23, I24, I30, K31, Q40, K46, K50, I63, and 164) were common between either the scFv or full-length KKO epitope (FIG. 8b). However, mutant B did appear to bind within a similar region on PF4 that overlaps with both full-length KKO and scFv binding sites, which is also recognized by pathogenic HIT antibodies¹⁹ (FIG. 8a). Interestingly, two amino acids in the mutant B epitope (H23 and K46) overlapped with the heparin-binding site on PF4.^{40,44} This site is also known to bind group 3/heparin-independent antibodies that are produced in combination with group 2/heparin-dependent antibodies by a subset of HIT-positive patients.⁴⁰ Mutant B similarly bound to two amino acids on PF4 (H23 and Q40) that were previously reported as critical for the binding of HIT patient sera that contained a polyclonal mixture of heparin-dependent and -independent antibodies.^{40,44}

Inhibiting HIT-Positive (EIA+/SRA+) HIT-Negative (EIA+/SRA−) antibody binding: Sera (n=10) from confirmed HIT-positive patients (EIA-positive/SRA-positive) containing anti-PF4/heparin antibodies were used in a modified streptavidin-biotin based anti-PF4/heparin IgG EIA (streptavidin anti-PF4/hep EIA) with scFv at 50 μg/mL. On average, antibody binding to PF4/heparin was reduced in the presence of scFv.

The ability of each scFv construct to inhibit antibody binding is shown as a decrease in absorbance (OD) at 405 nm where an OD405 nm≥0.45 in this assay is considered positive for anti-PF4/heparin antibodies. The weakest inhibitors in the streptavidin anti-PF4/hep EIA were wildtype scFv and mutant C, which also showed the worst performance in earlier characterization experiments. Only 2/10 (20.0%) HIT-positive patients became negative (OD405 nm≤0.45) with the addition of either wildtype scFv or mutant C at maximum concentrations, respectively (Table 5). scFv mutants D and E showed improved inhibition of pathogenic antibody binding, as 7/10 (70.0%) and 8/10 (80.0%) HIT-positive patients become negative in with the addition of either construct at 50 μg/mL, respectively (Table 5). The strongest inhibitors of pathogenic antibody binding in the streptavidin anti-PF4/hep EIA were scFv mutants B and F. When 50 μg/mL of either construct was added, 9/10 (90.0%) HIT-positive patients became negative and when 10 μg/mL was added, 7/10 (Mutant B, 70.0%) and 6/10 (Mutant F, 60.0%) HIT-positive patients became negative, further highlighting the strength of these constructs (Table 5).

A significantly greater difference between antibody binding (OD405 nm≥0.45) was also observed in all HIT-positive patients with or without the addition of wildtype scFv (1.657 and 0.931, p=0.018), mutant B (1.565 and 0.275, p=0.001), mutant C (1.684 and 0.799, p=0.010), mutant D (1.840 and 0.283, p=0.001), mutant E (1.682 and 0.319, p=0.001), and mutant F (1.723 and 0.281, p=0.002).

Sera (n=10) from confirmed HIT-negative patients (EIA-positive/SRA-negative) containing anti-PF4/heparin antibodies were then used in the same EIA with scFv constructs to determine if antibody binding was decreased in this cohort (Table 5). Neither wildtype nor mutant scFv constructs were able to significantly inhibit antibody binding to PF4/heparin at any concentration (Table 5). The mean OD405 nm at 50 μg/mL for all HIT-negative patients with and without the addition of wildtype scFv was 1.983 and 1.407 (p=0.303), mutant B was 0.893 and 1.102 (p=0.438), mutant C was 0.932 and 1.235 (p=0.285), mutant D was 0.910 and 1.002 (p=0.700), mutant E was 0.876 and 1.080 (p=0.430), and mutant F was 0.841 and 1.189 (p=0.195).

TABLE 5
Mean absorbance (optical density [OD] at 405 nm) of
anti-PF4/heparin antibodies from HIT-Positive (n = 10) and
HIT-negative (n = 10) patient sera.

| | HIT-Positive Sera | | Statistical |
|---|---|---|---|
| KKO-scFv | (Mean OD₄₀₅ₙₘ, n = 10) | | Significance |
| Construct | − scFv | + scFv | (p value) |
| Wildtype | 1.657 | 0.931 | 0.018 |
| Mutant B | 1.565 | 0.275 | 0.001 |
| Mutant C | 1.684 | 0.799 | 0.010 |
| Mutant D | 1.840 | 0.283 | 0.001 |
| Mutant E | 1.682 | 0.319 | 0.001 |
| Mutant F | 1.723 | 0.281 | 0.002 |
| | HIT-Negative Sera | | Statistical |
| KKO-scFv | (Mean OD₄₀₅ₙₘ, n = 10) | | Significance |
| Construct | − scFv | + scFv | (p value) |
| Wildtype | 1.083 | 1.407 | 0.303 |
| Mutant B | 0.893 | 1.102 | 0.438 |
| Mutant C | 0.932 | 1.235 | 0.285 |
| Mutant D | 0.910 | 1.002 | 0.700 |
| Mutant E | 0.876 | 1.080 | 0.430 |
| Mutant F | 0.841 | 1.189 | 0.195 |

Statistical significance (p-value) between anti-PF4/heparin antibody binding before (−) and after (+) the addition of 50 μg/mL wildtype or mutant scFv is indicated.

The percentage of inhibition for each scFv construct was also determined against sera from confirmed HIT-positive (n=10) and HIT-negative (n=10) patients at 50 μg/mL (Table 6). Better diagnostic discrimination between HIT-positive and HIT-negative patients in the streptavidin anti-PF4/hep EIA was observed in the presence of each scFv construct. Wildtype scFv inhibited HIT-positive and HIT-negative antibodies by an average percentage of 44.7% and −38.3% (p=0.0002), respectively (Table 6). Mutant B inhibited HIT-positive and HIT-negative antibodies by an average percentage of 77.8% and −31.4% (p<0.0001), respectively (Table 6). Mutant C inhibited HIT-positive and HIT-negative antibodies by an average percentage of 50.6% and −42.5% (p=0.0004), respectively (Table 6). Mutant D inhibited HIT-positive and HIT-negative antibodies by an average percentage of 78.3% and −17.3% (p<0.0001), respectively (Table 6). Mutant E inhibited HIT-positive and HIT-negative antibodies by an average percentage of 76.5% and −34.0% (p=0.0001), respectively (Table 6). Mutant F inhibited HIT-positive and HIT-negative antibodies by an average percentage of 80.0% and −57.0% (p=0.0005), respectively (Table 6). While each construct of scFv was able to decrease antibody binding to varying degrees, mutants B, D, E, and F remained the strongest inhibitors compared to wildtype. Furthermore, antibody binding in HIT-negative patient samples was not significantly altered in the presence of any scFv construct in this assay.

TABLE 6
Wildtype and mutant scFv mean percent (%) inhibition of
anti-PF4/heparin antibodies from HIT-Positive (n = 10) and
HIT-Negative (n = 10) patient sera at 50 μg/mL.

| | HIT-Positive Sera | HIT-Negative Sera | Statistical |
|---|---|---|---|
| | (% Inhibition) | (% Inhibition) | Significance |
| scFv Construct | (Mean, n = 10) | (Mean, n = 10) | (p value) |
| Wildtype | 44.7% | −38.3% | 0.0002 |
| Mutant B | 77.8% | −31.4% | <0.0001 |
| Mutant C | 50.6% | −42.5% | 0.0004 |
| Mutant D | 78.3% | −17.3% | <0.0001 |
| Mutant E | 76.5% | −34.0% | 0.0001 |
| Mutant F | 80.0% | −57.0% | 0.0005 |

Assessing the clinical applications of high-affinity scFv mutants. A large-scale screening of HIT patient sera was performed to further evaluate the utility of this assay as a diagnostic test. Based on previous characterization experiments, mutant B and mutant F were chosen as lead candidate mutants because they had stronger affinities in BLI, superior inhibitory activities against KKO full-length, and provided the best separation between pathogenic and non-pathogenic anti-PF4/heparin antibodies. Sera from confirmed HIT-positive (n=20) and HIT-negative (n=20) patients were then tested to further determine if mutant scFv can improve the diagnostic performance of the streptavidin anti-PF4/hep EIA by eliminating the high rate of false-positive results typically generated in this assay. Wildtype scFv was tested alongside mutant B and F scFv as a control to compare performances.

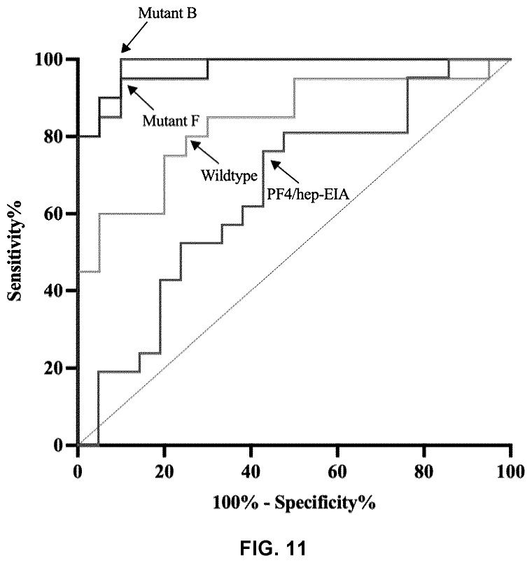

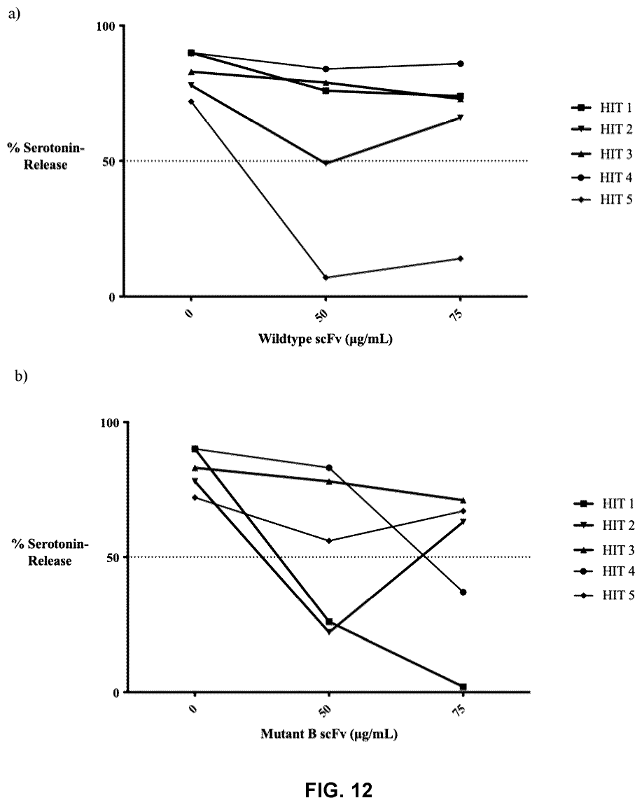

The ability of scFv to inhibit antibody binding at an optimal concentration of 50 μg/mL was determined based on the ability of these constructs to decrease antibody absorbance levels in a particular sample to negative threshold levels (OD405 nm≤0.45 nm). Levels of pathogenic and non-pathogenic anti-PF4/heparin antibody binding remained significantly different between the upscaled HIT-positive and HIT-negative patient cohorts before and after the addition of scFv wildtype (p=0.019), mutant B (p=0.0001), and mutant F (p=0.0007) (FIG. 9). Furthermore, the addition of either mutant B or mutant F to the EIA assay almost always blocked binding of anti-PF4/heparin antibodies (OD405 nm≤0.45 nm) in sera from HIT-positive patients, providing superior separation between pathogenic and non-pathogenic antibodies compared to wildtype scFv (FIG. 9). Only 4/20 (20.0%) and 6/20 (30.0%) patients remained positive in the presence of scFv mutant B or mutant F, respectively compared to 17/20 (85.0%) patients in the presence of wildtype scFv (FIG. 9). A significant difference between pathogenic and non-pathogenic anti-PF4/heparin antibody binding was also observed in the presence of mutant B (mean inhibition; 75.8% vs. −4.4%, p<0.0001, FIG. 10) and mutant F (mean inhibition; 76.0% vs. −13.0%, p<0.0001, FIG. 10).

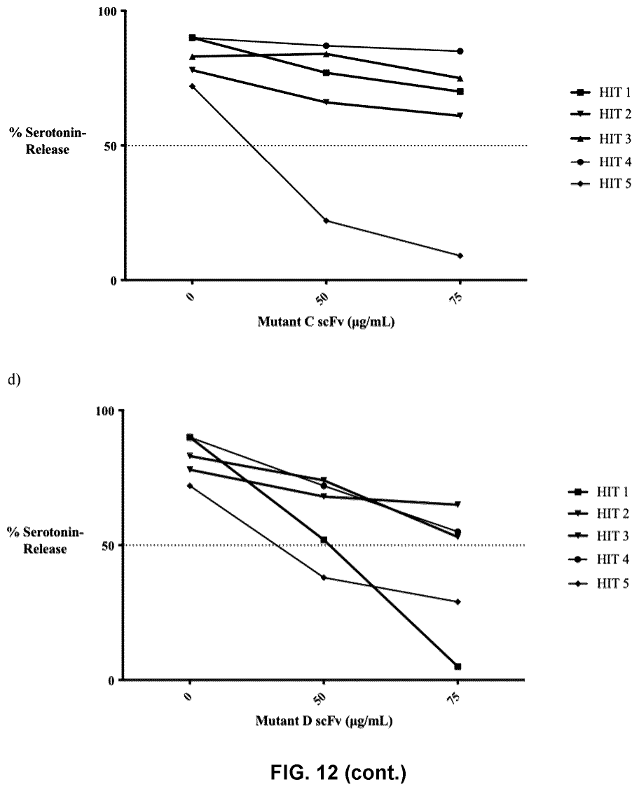

Determining new positivity thresholds and diagnostic performance of a novel HIT diagnostic assay. Using the previously tested patient samples (HIT-positive, n=20; HIT-negative, n=20), receiver operating characteristic (ROC) curves for the streptavidin anti-PF4/heparin IgG EIA using wildtype scFv, mutant B, or mutant F were generated to further assess the diagnostic performance of this assay (FIG. 11). The ideal measurement for this assay was determined to be the percentage of inhibition rather than absorbance levels because they provide a better diagnostic sensitivity and specificity (data not shown). Based on ROC curve analysis, the positivity thresholds for this assay when using scFv wildtype, mutant B, and mutant F was a percentage of inhibition greater or equal to (≥) 42.0%, ≥39.8%, and ≥54.6%, respectively. The area under the curve (AUC) value, which measures the ability of an assay to discriminate between patients with or without the disease,⁴⁵ was then determined. As shown in Table 7, scFv wildtype had an AUC value of 0.84 (95% Confidence Interval [CI] 0.71-0.97), mutant B had an AUC of 0.98 (95% CI 0.95-1.00), and mutant F had an AUC of 0.98 (95% CI 0.94-1.00).

TABLE 7
Area under the curve (AUC) and positivity threshold for the
streptavidin IgG-specific anti-PF4/heparin enzyme immunoassay
with (anti-PF4/hep-EIA) and without scFv (scFv anti-PF4/hep-EIA)

| | AUC | Positivity |
|---|---|---|
| Assay Type | (95% CI) | Threshold |
| Anti-PF4/hep-EIA | 0.66 | OD₄₀₅ₙₘ ≥ 0.8 |
| | (0.49 to 0.83) | |
| WT scFv anti-PF4/hep-EIA | 0.84 | ≥42.0% Inhibition |
| | (0.71 to 0.97) | |
| Mutant B scFv anti-PF4/hep-EIA | 0.98 | ≥39.8% Inhibition |
| | (0.95 to 1.00) | |
| Mutant F scFv anti-PF4/hep-EIA | 0.98 | ≥54.6% Inhibition |
| | (0.94 to 1.00) | |

CI = Confidence Interval

The sensitivity and specificities of this assay were then determined using each scFv construct at the ROC determined optimal thresholds in a patient cohort specifically curated and with a disease prevalence of 50% (Table 8). In this cohort, the EIA at positivity threshold of OD₄₀₅ₙₘ≥0.8 had a sensitivity and specificity of 81.0% (60.0-92.3%) and 52.4% (32.4-71.7%), respectively with a positive predictive value (PPV) of 63.0% (50.9-73.6%) and negative predictive value (NPV) of 73.3% (51.0-87.9%). The EIA with wildtype scFv at a positivity threshold of ≥42.0% inhibition had a sensitivity and specificity of 70% (95% CI 45.7-88.1) and 95% (75.1-99.9), respectively with a positive predictive value (PPV) of 93.3% (95% CI 67.0-99.0) and negative predictive value (NPV) of 76.0% (95% CI 61.7-86.2). When using Mutant B at a positivity threshold of ≥39.8% inhibition, this assay had a sensitivity and specificity of the EIA was 100.0% and 90.0% (95% CI 68.3-88.8), respectively with a PPV of 90.9% (95% CI 72.8-97.4) and NPV of 100.0%. Lastly, the addition of mutant F in this assay at a positivity threshold of ≥54.6% inhibition had a sensitivity and specificity of 95.0% (95% CI 75.1-99.9) and 90.0% (95% CI 68.3-98.8), respectively with a PPV of 90.5% (95% CI 71.8-97.3) and NPV of 94.7% (95% CI 79.6-98.4). Therefore, mutant scFv was not only able to distinguish between pathogenic and non-pathogenic anti-PF4/heparin antibodies, but significantly improved the specificity (90.0%) of the streptavidin anti-PF4/hep EIA without sacrificing sensitivity (95.0-100.0%) compared to previous reports.^{8,9,29,30}

TABLE 8
Performance characteristics of a streptavidin IgG-specific anti-PF4/
heparin inhibitory EIA with (scFv anti-PF4/hep-EIA) and without
scFv in a pre-screened patient cohort with a disease prevalence of 50%.

| | Sensitivity | Specificity | PPV | NPV |
|---|---|---|---|---|
| Assay Type | (95% CI) | (95% CI) | (95% CI) | (95% CI) |
| Streptavidin anti- | 81.0% | 52.4% | 63.0% | 73.3% |
| PF4/hep EIA | (60.0- | (32.4- | (50.9- | (51.0- |
| | 92.3%) | 71.7%) | 73.6%) | 87.9%) |
| Wildtype scFv | 70.0% | 95.0% | 93.3% | 76.0% |
| Streptavidin anti- | (45.7- | (75.1- | (67.0- | (61.7- |
| PF4/hep EIA | 88.1%) | 99.9%) | 99.0%) | 86.2%) |
| Mutant B scFv | 100.0% | 90.0% | 90.9% | 100.0% |
| Streptavidin anti- | (83.2- | (68.3- | (72.9- | |
| PF4/hep EIA | 100.0%) | 98.8%) | 97.4%) | |
| Mutant F scFv | 95.0% | 90.0% | 90.5% | 94.7% |
| Streptavidin anti- | (75.1- | (68.3- | (71.8- | (79.6- |
| PF4/hep EIA | 99.9%) | 98.8%) | 97.3%) | 98.4%) |

CI = Confidence Interval

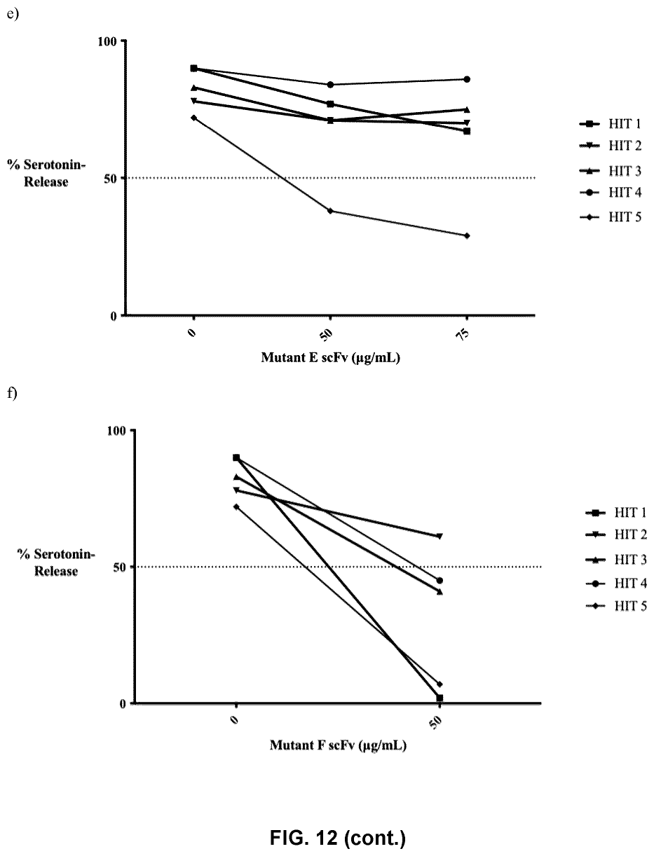

Inhibiting immune complex-mediated platelet activation. Experiments were then performed to determine if wildtype and mutant scFv can inhibit the functional activity of pathogenic antibodies in an SRA. As shown in Table 9, the ability of each scFv variant to inhibit platelet activation resulting in a moderate (≥20%) or strong (≥50%) decrease in the percent of ¹⁴C-serotonin release was tested using strongly reactive (¹⁴C-serotonin release >85%) HIT-positive (EIA+/SRA+) patient sera (FIG. 12).

TABLE 9
Percent (%) inhibition of serotonin release. HIT-positive patients (n = 5) were tested
in the C¹⁴-serotonin release assay (SRA) in the presence of 50 μg/mL and 75 μg/mL
wildtype or mutant scFv to determine if these constructs can inhibit platelet activation
by anti-PF4/heparin antibodies. Percentage of inhibition was calculated by determining
the baseline serotonin-release of patient sample in the SRA without any scFv added.

| Construct | | | | | | | | | | |
|---|---|---|---|---|---|---|---|---|---|---|
| (scFv) | HIT-Positive | | HIT-Positive | | HIT-Positive | | HIT-Positive | | HIT-Positive | |
| [conc] | Patient #1 | | Patient #2 | | Patient #3 | | Patient #4 | | Patient #5 | |
| (μg/mL | Inhibition (%) | | Inhibition (%) | | Inhibition (%) | | Inhibition (%) | | Inhibition (%) | |
| scFv) | 50 | 75 | 50 | 75 | 50 | 75 | 50 | 75 | 50 | 75 |
| Wildtype | 15.6 | 17.8 | 37.2 | 15.4 | 4.8 | 12.0 | 6.7 | 4.4 | −6.9 | 20.8 |
| Mutant B | 71.1 | 97.8 | 71.8 | 19.2 | 6.0 | 14.5 | 7.8 | 58.9 | 90.3 | 80.6 |
| Mutant C | 14.4 | 22.2 | 15.4 | 21.8 | −1.2 | 9.6 | 3.3 | 5.6 | 22.2 | 6.9 |
| Mutant D | 42.2 | 94.4 | 12.8 | 16.7 | 10.8 | 36.1 | 20.0 | 38.9 | 69.4 | 87.5 |
| Mutant E | 14.4 | 25.6 | 9.0 | 10.3 | 14.5 | 9.6 | 6.7 | 4.4 | 47.2 | 59.7 |
| Mutant F | 97.8 | N/A | 21.8 | N/A | 50.6 | N/A | 50.0 | N/A | 90.3 | N/A |

Baseline platelet activation was assessed before the addition of any construct and in the presence of wildtype or mutant scFv at 50 and 75 μg/mL concentrations (FIG. 12). Wildtype scFv inhibited platelet activation moderately in 2/5 (40%) HIT-positive samples and strongly inhibited platelet activation in 0/5 (0.0%) samples at both 50 and 70 μg/mL concentrations (FIG. 12a). Comparatively, mutant B strongly inhibited platelet activation in 4/5 (80.0%) HIT-positive samples at either one or both concentrations (FIG. 12b). Mutant C performed similarly to wildtype, moderately inhibiting 3/5 (60.0%) HIT-positive samples and strongly inhibiting 0/5 (0.0%) HIT-positive samples at all concentrations (FIG. 12c). At one or both concentrations, mutant D inhibited platelet activation moderately in 3/5 (60.0%) HIT-positive samples and strongly in 2/5 (40%) HIT-positive samples (FIG. 12d). Platelet activation was moderately inhibited by mutant E in 1/5 (20.0%) HIT-positive samples and strongly inhibited in 1/5 (20.0%) HIT-positive samples at 75 μg/mL (FIG. 12e). Although mutant F was only tested at one concentration (50 μg/mL) due to insufficient sample volume, this construct was able to strongly inhibit platelet activation in 3/5 (60.0%) HIT-positive samples (FIG. 12f).

These findings demonstrate platelet activation was inhibited to varying degrees with each patient sample by different scFv constructs. However, mutants B and F caused the largest decrease in platelet activation when compared to other constructs, aligning with findings from previous experiments that further highlight the improved performance of these mutants.

While the present application has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### Sequences

S1 Sense Primer (SEQ ID NO: 1)
GTTATTACTCGCGGCCCAGCCGGCCATGGCTGATATCCAGATGATCCAGA
GCC
AS1 Anti-Sense Primer (SEQ ID NO: 2)
CCATGATGGTGGTGATGGTGTTGGCCTCCCGGGCCACTAGTGGCAGACAC
GGTAACCAGG
For SEQ ID NOs: 3-8, italics indicate GS-linker peptide, separating N-terminal variable
light chain region from C-terminal variable heavy chain region; underlined amino acids
indicate CDR regions, and bolded residues indicate a mutation from the original
sequence
Wildtype scFv amino acid sequence (SEQ ID NO: 3)
DIQMIQSQKFMSTSVGDRVTVTCKASQNVGTNVAWYQQKPGQSPNALIYSA
SYRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTNYFIY
WVKQRPGQGLEWIGEINPRNGDTDFNEKFESRATLTVDKSSSTAYMQLSSLT
SEDSAIYYCTRSPYGNNYGFTYWGQGTLVTVSA
Mutant B scFv amino acid sequence (SEQ ID NO: 4)
DIQMIQSQKFMSTSVGDKVTVTCKASQNVGTNVAWYQQKPGQSPNALIYSA
SYRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTKYFIY
WVKQRPGQGLEWIGEINPRNGDTNFNEKFESRATLTVDKSSSTAYMQLSSLT
SEDSAIYYCTRSPYGNNYGFTYWGQGTLVTVSA
Mutant C scFv amino acid sequence (SEQ ID NO: 5)
DIQMIQSQKFMSTSVGDRVTVTCKASQNVGTNVAWYQQKPGQSPNALIYNA
SHRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTNYFIY
WVKQRPGQGLEWIGEINPRNGDTDFNEKFESRATLTVDKSSSTAYMQLSSLT
SEDSAIYYCTRSPYGNNYGFTYWGQGTLVTVSA
Mutant D scFv amino acid sequence (SEQ ID NO: 6)
DIQMIQSQKFMSTSVGDRVTVTCKASQNVGTNVAWYQQKPGQSPNALIYSA
SYRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTNYFIH
WVKQRPGQGLEWIGEINPRNGDTDFNEKFESRATLTVDKSSSTAYMQLSSLT
SEDSAIYYCTRSPYGNNYGFTYWGQGTLVTVSA
Mutant E scFv amino acid sequence (SEQ ID NO: 7)
DIQMIQSQKFMSTSVGDRVTVTCKASQNVGTNVAWYQQKPGQSPNALIYSA
SYRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTNYFIH
WVKQRPGQGLEWIGEINPKNGDTGFNEKFESRATLTVDKSSSTAYMQLSSLT
SEDSAIYYCTRSPYGNNYGFTYWGPGTLVTVSA
Mutant F scFv amino acid sequence (SEQ ID NO: 8)
DIQMIQSQKFMSTSVGDRVTVTCKASQNVGTNVAWYQQKPGQSPNALIYSA
SYRYSGVPDRFTGSGSGTDFTLTITNVQSEDLADYFCQQYNSYPLTFGTGTKL
DLKGGGGSGGGGSGGGGSVQLQQSGAELVKPGASVKLSCKASGYTFTNYFIY
WVKQRPGQGLEWIGEINPRNGDTDFNVKFKSRATLTVDKSSSTAYMQLSSLT
SEDSAIYYCTRSPYRNNYGFTYWGQGTLVTVSA
Wildtype variable light chain (SEQ ID NO: 9)
DIQMIQSQKFMSTSVGDRVTVTCKASQNVGTNVAWYQQKPGQSPNALIYSA
SYRYSGVPDRFTGSGSGTDFTLTITNVQSEDLADYFCQQYNSYPLTFGTGTKL
DLK
Wildtype variable heavy chain (SEQ ID NO: 10)
VQLQQSGAELVKPGASVKLSCKASGYTFTNYFIYWVKQRPGQGLEWIGEINP
RNGDTDFNEKFESRATLTVDKSSSTAYMQLSSLTSEDSAIYYCTRSPYGNNYG
FTYWGQGTLVTVSA
Mutant B variable light chain (SEQ ID NO: 11)
DIQMIQSQKFMSTSVGDKVTVTCKASQNVGTNVAWYQQKPGQSPNALIYSA
SYRYSGVPDRFTGSGSGTDFTLTITNVQSEDLADYFCQQYNSYPLTFGTGTKL
DLK
Mutant B variable heavy chain (SEQ ID NO: 12)
VQLQQSGAELVKPGASVKLSCKASGYTFTKYFIYWVKQRPGQGLEWIGEINP
RNGDTNFNEKFESRATLTVDKSSSTAYMQLSSLTSEDSAIYYCTRSPYGNNYG
FTYWGQGTLVTVSA
Mutant C variable light chain (SEQ ID NO: 13)
DIQMIQSQKFMSTSVGDRVTVTCKASQNVGTNVAWYQQKPGQSPNALIYNA
SHRYSGVPDRFTGSGSGTDFTLTITNVQSEDLADYFCQQYNSYPLTFGTGTKL
DLK
Mutant D variable heavy chain (SEQ ID NO: 14)
VQLQQSGAELVKPGASVKLSCKASGYTFTNYFIHWVKQRPGQGLEWIGEINP
RNGDTDFNEKFESRATLTVDKSSSTAYMQLSSLTSEDSAIYYCTRSPYGNNYG
FTYWGQGTLVTVSA
Mutant E variable heavy chain (SEQ ID NO: 15)
VQLQQSGAELVKPGASVKLSCKASGYTFTNYFIHWVKQRPGQGLEWIGEINP
KNGDTGFNEKFESRATLTVDKSSSTAYMQLSSLTSEDSAIYYCTRSPYGNNYG
FTYWGPGTLVTVSA
Mutant F variable heavy chain (SEQ ID NO: 16)
VQLQQSGAELVKPGASVKLSCKASGYTFTNYFIYWVKQRPGQGLEWIGEINP
RNGDTDFNVKFKSRATLTVDKSSSTAYMQLSSLTSEDSAIYYCTRSPYRNNY
GFTYWGQGTLVTVSA
Human PF4 (CXCL4) (SEQ ID NO: 17)
EAEEDGDLQCLCVKTTSQVRPRHITSLEVIKAGPHCPTAQLIATLKNGRKICLD
LQAPLYKKIIKKLLES
CDR L1 Wildtype (SEQ ID NO: 18)
KASQNVGTNVA
CDR L2 Wildtype (SEQ ID NO: 19)
SASYRYS
CDR L3 Wildtype (SEQ ID NO: 20)
QQYNSYPLT
CDR H1 Wildtype (SEQ ID NO: 21)
NYFIY
CDR H2 Wildtype (SEQ ID NO: 22)
EINPRNGDTDFNEKFES
CDR H3 Wildtype (SEQ ID NO: 23)
SPYGNNYGFTY
CDR H1 Mutant B (SEQ ID NO: 24)
KYFIY
CDR H2 Mutant B (SEQ ID NO: 25)
EINPRNGDTNFNEKFES
CDR L2 Mutant C (SEQ ID NO: 26)
NASHRYS
CDR H1 Mutant D, E (SEQ ID NO: 27)
NYFIH
CDR H2 Mutant E (SEQ ID NO: 28)
EINPKNGDTGFNEKFES
CDR H2 Mutant F (SEQ ID NO: 29)
EINPRNGDTDFNVKFKS
CDR H3 Mutant F (SEQ ID NO: 30)
SPYRNNYGFTY
linker (SEQ ID NO: 31)
GGGGSGGGGSGGGGS
Wildtype scFv nucleic acid sequence (SEQ ID NO: 32)
GATATCCAGATGATCCAGAGCCAGAAATTCATGAGCACCAGCGTGGGCGACA
GAGTGACCGTGACATGTAAAGCCAGCCAGAACGTGGGCACCAACGTGGCCTGGT
ATCAGCAGAAGCCTGGACAGAGCCCCAACGCTCTGATCTACAGCGCCAGCTACA
GATACAGCGGCGTGCCCGATAGATTCACAGGCAGCGGCTCTGGCACCGACTTCAC
CCTGACCATCACCAATGTGCAGAGCGAGGACCTGGCCGACTACTTCTGCCAGCAG
TACAACAGCTACCCTCTGACCTTTGGCACCGGCACCAAGCTGGATCTTAAAGGCG
GCGGAGGATCTGGCGGAGGTGGAAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAACTACTTCATCTACTGGGTCAAGCAGCGGCCAGGC
CAGGGACTCGAATGGATCGGAGAGATCAACCCCAGAAACGGCGACACCGATTTC
AACGAGAAGTTCGAGAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACGGCAACAACTACGGCTTTACCTATTGGGGCCAGGGCACCCT
GGTTACCGTGTCTGCC
Mutant B scFv nucleic acid sequence (SEQ ID NO: 33)
GATATCCAGATGATCCAGAGCCAGAAATTCATGAGCACCAGCGTGGGCGACA
AAGTGACCGTGACATGTAAAGCCAGCCAGAACGTGGGCACCAACGTGGCCTGGT
ATCAGCAGAAGCCTGGACAGAGCCCCAACGCTCTGATCTACAGCGCCAGCTACA
GATACAGCGGCGTGCCCGATAGATTCACAGGCAGCGGCTCTGGCACCGACTTCAC
CCTGACCATCACCAATGTGCAGAGCGAGGACCTGGCCGACTACTTCTGCCAGCAG
TACAACAGCTACCCTCTGACCTTTGGCACCGGCACCAAGCTGGATCTTAAAGGCG
GCGGAGGATCTGGCGGAGGTGGAAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAAGTACTTCATCTACTGGGTCAAGCAGCGGCCAGGC
CAGGGACTCGAATGGATCGGAGAGATCAACCCCAGAAACGGCGACACCAATTTC
AACGAGAAGTTCGAGAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACGGCAACAACTACGGCTTTACCTATTGGGGCCAGGGCACCCT
GGTTACCGTGTCTGCC
Mutant C scFv nucleic acid sequence (SEQ ID NO: 34)
GATATCCAGATGATCCAGAGCCAGAAATTCATGAGCACCAGCGTGGGCGACA
GAGTGACCGTGACATGTAAAGCCAGCCAGAACGTGGGCACCAACGTGGCCTGGT
ATCAGCAGAAGCCTGGACAGAGCCCCAACGCTCTGATCTACAACGCCAGCCACA
GATACAGCGGCGTGCCCGATAGATTCACAGGCAGCGGCTCTGGCACCGACTTCAC
CCTGACCATCACCAATGTGCAGAGCGAGGACCTGGCCGACTACTTCTGCCAGCAG
TACAACAGCTACCCTCTGACCTTTGGCACCGGCACCAAGCTGGATCTTAAAGGCG
GCGGAGGATCTGGCGGAGGTGGAAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAACTACTTCATCTACTGGGTCAAGCAGCGGCCAGGC
CAGGGACTCGAATGGATCGGAGAGATCAACCCCAGAAACGGCGACACCGATTTC
AACGAGAAGTTCGAGAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACGGCAACAACTACGGCTTTACCTATTGGGGCCAGGGCACCCT
GGTTACCGTGTCTGCC
Mutant D scFv nucleic acid sequence (SEQ ID NO: 35)
GATATCCAGATGATCCAGAGCCAGAAATTCATGAGCACCAGCGTGGGCGACA
GAGTGACCGTGACATGTAAAGCCAGCCAGAACGTGGGCACCAACGTGGCCTGGT
ATCAGCAGAAGCCTGGACAGAGCCCCAACGCTCTGATCTACAGCGCCAGCTACA
GATACAGCGGCGTGCCCGATAGATTCACAGGCAGCGGCTCTGGCACCGATTTCAC
CCTGACCATCACCAATGTGCAGAGCGAGGACCTGGCCGACTACTTCTGCCAGCAG
TACAACAGCTATCCTCTGACCTTTGGCACCGGCACCAAGCTGGATCTTAAAGGCG
GCGGAGGATCTGGCGGAGGTGGAAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAACTACTTCATCCACTGGGTCAAGCAGCGGCCAGGC
CAGGGACTCGAATGGATAGGAGAGATCAACCCCAGAAACGGCGACACCGATTTC
AACGAGAAGTTCGAGAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACGGCAACAACTACGGCTTTACCTATTGGGGCCAGGGCACCCT
GGTTACCGTGTCTGCC
Mutant E scFv nucleic acid sequence (SEQ ID NO: 36)
GATATCCAGATGATCCAGAGCCAGAAATTCATGAGCACCAGCGTGGGCGACA
GAGTGACCGTGACATGTAAAGCCAGCCAGAACGTGGGCACCAACGTGGCCTGGT
ATCAGCAGAAGCCTGGACAGAGCCCCAACGCTCTGATCTACAGCGCCAGCTACA
GATACAGCGGCGTGCCCGATAGATTCACAGGCAGCGGCTCTGGCACCGACTTCAC
CCTGACCATCACCAATGTGCAGAGCGAGGACCTGGCCGACTACTTCTGCCAGCAG
TACAACAGCTACCCTCTGACCTTTGGCACCGGCACCAAGCTGGATCTTAAAGGCG
GCGGAGGATCTGGCGGAGGTGGTAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAACTACTTCATCCACTGGGTCAAGCAGCGGCCAGGC
CAGGGACTCGAATGGATCGGAGAGATCAACCCCAAAAACGGCGACACCGGTTTC
AACGAGAAGTTCGAGAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACGGCAACAACTACGGCTTTACCTATTGGGGCCCGGGCACCCT
GGTTACCGTGTCTGCC
Mutant F scFv nucleic acid sequence (SEQ ID NO: 37)
GATATCCAGATGATCCAGAGCCAGAAATTCATGAGCACCAGCGTGGGCGACA
GAGTGACCGTGACATGTAAAGCCAGCCAGAACGTGGGCACCAACGTGGCCTGGT
ATCAGCAGAAGCCTGGACAGAGCCCCAACGCTCTGATCTACAGCGCCAGCTACA
GATACAGCGGCGTGCCCGATAGATTCACAGGCAGCGGCTCTGGCACCGACTTCAC
CCTGACCATCACCAATGTGCAGAGCGAGGACCTGGCCGACTACTTCTGCCAGCAG
TACAACAGCTACCCTCTGACCTTTGGCACCGGCACCAAGCTGGATCTTAAAGGCG
GCGGAGGATCTGGCGGAGGTGGAAGCGGAGGCGGTGGATCTGTTCAGCTGCAAC
AATCTGGCGCCGAGCTGGTTAAGCCTGGCGCCTCTGTGAAGCTGAGCTGCAAGGC
CAGCGGCTACACCTTCACCAACTACTTCATCTACTGGGTCAAGCAGCGGCCAGGC
CAGGGACTCGAATGGATCGGAGAGATCAACCCCAGAAACGGCGACACCGATTTC
AACGTGAAGTTCAAAAGCCGGGCCACACTGACCGTGGATAAGTCTAGCAGCACC
GCCTACATGCAGCTGAGCAGCCTGACAAGCGAGGACTCCGCCATCTACTACTGCA
CAAGAAGCCCCTACCGCAACAACTACGGCTTTACCTATTGGGGCCAGGGCACCCT
GGTTACCGTGTCTGCC

### REFERENCES

1. Warkentin T E, Sheppard J I, Sun J C, Jung H, Eikelboom J W. Anti-PF4/heparin antibodies and venous graft occlusion in postcoronary artery bypass surgery patients randomized to postoperative unfractionated heparin or fondaparinux thromboprophylaxis. J Thromb Haemost 2013; 11(2): 253-60.
2. Gerotziafas G T, Elalamy I, Lecrubier C, et al. The role of platelet factor 4 in platelet aggregation induced by the antibodies implicated in heparin-induced thrombocytopenia. Blood Coagul Fibrinolysis 2001; 12(7): 511-20.
3. Cines D B, Rauova L, Arepally G, et al. Heparin-induced thrombocytopenia: an autoimmune disorder regulated through dynamic autoantigen assembly/disassembly. J Clin Apher 2007; 22(1): 31-6.
4. Trossaert M, Gaillard A, Commin P L, Amiral J, Vissac A M, Fressinaud E. High incidence of anti-heparin/platelet factor 4 antibodies after cardiopulmonary bypass surgery. Br J Haematol 1998; 101(4): 653-5.
5. Selleng S, Selleng K, Wollert H G, et al. Heparin-induced thrombocytopenia in patients requiring prolonged intensive care unit treatment after cardiopulmonary bypass. J Thromb Haemost 2008; 6(3): 428-35.
6. Warkentin T E, Sheppard J A, Horsewood P, Simpson P J, Moore J C, Kelton J G. Impact of the patient population on the risk for heparin-induced thrombocytopenia. Blood 2000; 96(5): 1703-8.
7. Nazi I, Arnold D M, Warkentin T E, Smith J W. Staibano P. Kelton J G. Distinguishing between anti-platelet factor 4/heparin antibodies that can and cannot cause heparin-induced thrombocytopenia. J Thromb Haemost 2015; 13(10): 1900-7.
8. Warkentin T E, Sheppard J I, Moore J C, Sigouin C S, Kelton J G. Quantitative interpretation of optical density measurements using PF4-dependent enzyme-immunoassays. J Thromb Haemost 2008; 6(8): 1304-12.
9. Warkentin T E. Laboratory diagnosis of heparin-induced thrombocytopenia. Int J Lab Hematol 2019; 41 Suppl 1: 15-25.
10. Nazi I, Arnold D M, Moore J C, et al. Pitfalls in the diagnosis of heparin-Induced thrombocytopenia: A 6-year experience from a reference laboratory. Am J Hematol 2015; 90(7): 629-33.
11. Lo G K, Sigouin C S, Warkentin T E. What is the potential for overdiagnosis of heparin-induced thrombocytopenia? Am J Hematol 2007; 82(12): 1037-43.
12. Lee G M, Arepally G M. Diagnosis and management of heparin-induced thrombocytopenia. Hematol Oncol Clin North Am 2013; 27(3): 541-63.
13. Cuker A, Cines D B. How I treat heparin-induced thrombocytopenia. Blood 2012; 119(10): 2209-18.
14. Warkentin T E, Arnold D M, Nazi I, Kelton J G. The platelet serotonin-release assay. Am J Hematol 2015; 90(6): 564-72.
15. Caton S, O'Brien E, Pannelay A J, Cook R G. Assessing the clinical and cost impact of on-demand immunoassay testing for the diagnosis of heparin induced thrombocytopenia. Thromb Res 2016; 140: 155-62.
16. Suh J S. Aster R H, Visentin G P. Antibodies from patients with heparin-induced thrombocytopenia/thrombosis recognize different epitopes on heparin: platelet factor 4. Blood 1998; 91(3): 916-22.
17. Li Z Q, Liu W, Park K S, et al. Defining a second epitope for heparin-induced thrombocytopenia/thrombosis antibodies using KKO, a murine HIT-like monoclonal antibody. Blood 2002; 99(4): 1230-6.
18. Arepally G M, Kamei S, Park K S, et al. Characterization of a murine monoclonal antibody that mimics heparin-induced thrombocytopenia antibodies. Blood 2000; 95(5): 1533-40).
19. Huynh A, Arnold D M, Kelton J G, et al. Characterization of platelet factor 4 amino acids that bind pathogenic antibodies in heparin-induced thrombocytopenia. J Thromb Haemost 2019; 17(2): 389-99.
20. Cuker A. Rux A H, Hinds J L, et al. Novel diagnostic assays for heparin-induced thrombocytopenia. Blood 2013; 121(18): 3727-32.
21. Warkentin T E, Kelton J G. A 14-year study of heparin-induced thrombocytopenia. Am J Med 1996; 101(5): 502-7.
22. Greinacher A, Gopinadhan M, Gunther J U, et al. Close approximation of two platelet factor 4 tetramers by charge neutralization forms the antigens recognized by HIT antibodies. Arterioscler Thromb Vase Biol 2006; 26(10): 2386-93.
23. Rauova L. Poncz M, Mckenzie S E, et al. Ultralarge complexes of PF4 and heparin are central to the pathogenesis of heparin-induced thrombocytopenia. Blood 2005; 105(1): 131-8.
24. Warkentin T E, Sheppard J I. Generation of platelet-derived microparticles and procoagulant activity by heparin-induced thrombocytopenia IgG/serum and other IgG platelet agonists: a comparison with standard platelet agonists. Platelets 1999; 10(5): 319-26.
25. Warkentin T E, Hayward C P, Boshkov L K, et al. Sera from patients with heparin-induced thrombocytopenia generate platelet-derived microparticles with procoagulant activity: an explanation for the thrombotic complications of heparin-induced thrombocytopenia. Blood 1994; 84(11): 3691-9.
26. Amiral J. Bridey F. Wolf M, et al. Antibodies to macromolecular platelet factor 4-heparin complexes in heparin-induced thrombocytopenia: a study of 44 cases. Thromb Haemost 1995; 73(1): 21-8.
27. Visentin G P, Malik M, Cyganiak K A, Aster R H. Patients treated with unfractionated heparin during open heart surgery are at high risk to form antibodies reactive with heparin: platelet factor 4 complexes. J Lab Clin Med 1996; 128(4): 376-83.
28. Nguyen T H, Greinacher A. Distinct Binding Characteristics of Pathogenic Anti-Platelet Factor-4/Polyanion Antibodies to Antigens Coated on Different Substrates: A Perspective on Clinical Application. ACS Nano 2018; 12(12): 12030-41.
29. Husseinzadeh H D, Gimotty P A, Pishko A M, Buckley M, Warkentin T E, Cuker A. Diagnostic accuracy of IgG-specific versus polyspecific enzyme-linked immunoassays in heparin-induced thrombocytopenia: a systematic review and meta-analysis. J Thromb Haemost 2017; 15(6): 1203-12.
30. Nagler M, Bachmann LM, ten Cate H, ten Cate-Hoek A. Diagnostic value of immunoassays for heparin-induced thrombocytopenia: a systematic review and meta-analysis. Blood 2016; 127(5): 546-57.
31. Warkentin T E, Sheppard J I, Linkins L A, Arnold D M, Nazy I. Performance characteristics of an automated latex immunoturbidimetric assay [HemosIL® HIT-Ab (PF4-H)] for the diagnosis of immune heparin-induced thrombocytopenia. Thromb Res 2017; 153: 108-17.
32. Refaai M A, Conley G, Ortel T L, Francis J L. Evaluation of a rapid and automated heparin-induced thrombocytopenia immunoassay. Int J Lab Hematol 2019; 41(4): 478-84.
33. Meyer O, Salama A, Pittet N, Schwind P. Rapid detection of heparin-induced platelet antibodies with particle gel immunoassay (ID-HPF4). Lancet 1999; 354(9189): 1525-6.
34. Warkentin T E, Sheppard J I, Smith J W, et al. Combination of two complementary automated rapid assays for diagnosis of heparin-induced thrombocytopenia (HIT). J Thromb Haemost 2020; 18(6): 1435-46.
35. Althaus K, Hron G, Strobel U, et al. Evaluation of automated immunoassays in the diagnosis of heparin induced thrombocytopenia. Thromb Res 2013; 131(3): e85-90.
36. Barbas C F, Burton, D. R., Scott, J. K. & Silverman, G. J. Phage Display: A Laboratory Manual: Cold Springs Harbor Laboratory Press; 2001.
37. Kretz C A. Mapping the Substrate Recognition Landscapes of Metalloproteases Using Comprehensive Mutagenesis. Methods Mol Biol 2017; 1579: 209-28.
38. Huynh A, Arnold D M, Moore J C, Smith J W, Kelton J G, Nazy I. Development of a high-yield expression and purification system for platelet factor 4. Platelets 2018; 29(3): 249-56.
39. Huynh A, Arnold D M, Smith J W, et al. The role of fluid-phase immune complexes in the pathogenesis of heparin-induced thrombocytopenia. Thromb Res 2020; 194: 135-41.
40. Huynh A, Kelton J G, Arnold D M, Daka M, Nazy I. Antibody epitopes in vaccine-induced immune thrombotic thrombocytopaenia. Nature 2021; 596(7873): 565-9.
41. Kelton J G, Sheridan D, Santos A, et al. Heparin-induced thrombocytopenia: laboratory studies. Blood 1988; 72(3): 925-30.
42. Clark L A, Ganesan S, Papp S, van Vlijmen H W. Trends in antibody sequence changes during the somatic hypermutation process. J Immunol 2006; 177(1): 333-40.
43. Cai Z. Yarovoi S V, Zhu Z, et al. Atomic description of the immune complex involved in heparin-induced thrombocytopenia. Nat Commun 2015; 6: 8277.
44. Krauel K, Weber C, Brandt S, et al. Platelet factor 4 binding to lipid A of Gram-negative bacteria exposes PF4/heparin-like epitopes. Blood 2012; 120(16): 3345-52.
45. Mandrekar J N. Receiver operating characteristic curve in diagnostic test assessment. J Thorac Oncol 2010; 5(9): 1315-6.

1. An isolated anti-PF4 antibody which specifically binds an epitope of PF4, wherein the antibody binds PF4 and/or a PF4/heparin complex with at least or about 2-fold, at least or about 3-fold, at least or about 4-fold, at least or about 5-fold, at least or about 10-fold, at least or about 100-fold, or more than 100-fold greater affinity than an scFv having an amino acid sequence of SEQ ID NO: 3 as determined by Biolayer Interferometry (BLI).

2. The antibody of claim 1, comprising a light chain variable (VL) domain and a heavy chain variable (VH) domain, the VL domain comprising complementarity determining regions (CDRs) CDR-L1, CDR-L2, and CDR-L3, and the VH domain comprising CDRs CDR-H1, CDR-H2, and CDR-H3, wherein the amino acid sequences of said CDRs are as shown in any one of a), b), c), d), or e):
a)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 24
KYFIY;
CDR-H2
SEQ ID NO: 25
EINPRNGDTNFNEKFES;
and
CDR-H3
SEQ ID NO: 23
SPYGNNYGFTY;
b)
CDR-L1
SEQ ID NO: 18
KASQNVGTNVA;
CDR-L2
SEQ ID NO: 26
NASHRYS;
CDR-L3
SEQ ID NO: 20
QQYNSYPLT;
CDR-H1
SEQ ID NO: 21
NYFIY
CDR-H2
SEQ ID NO: 22
EINPRNGDTDFNEKFES
and
CDR-H3
SEQ ID NO: 23
SPYGNNYGFTY;
c)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 22
EINPRNGDTDFNEKFES
and
CDR-H3
SEQ ID NO: 23
SPYGNNYGFTY;
d)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 27
NYFIH;
CDR-H2
SEQ ID NO: 28
EINPKNGDTGFNEKFES;
and
CDR-H3
SEQ ID NO: 23
SPYGNNYGFTY;
or
e)
CDR-L1
SEQ ID NO: 18
KASQNVGTNVA;
CDR-L2
SEQ ID NO: 19
SASYRYS;
CDR-L3
SEQ ID NO: 20
QQYNSYPLT;
CDR-H1
SEQ ID NO: 21
NYFIY
CDR-H2
SEQ ID NO: 29
EINPRNGDTDFNVKFKS;
and
CDR-H3
SEQ ID NO: 30
SPYRNNYGFTY.

3. The antibody of claim 2, wherein the VL domain and VH domain comprise i) a polypeptide having an amino acid sequence of a) SEQ ID NOs: 11 and 12; b) SEQ ID NOs: 13 and 10; c) SEQ ID NOs: 9 and 14; d) SEQ ID NOs: 9 and 15; or e) SEQ ID NOs: 9 and 16; ii) a polypeptide having an amino acid sequence with at least 80%, at least 90%, or at least 95% sequence identity to a) SEQ ID NOs: 11 and 12; b) SEQ ID NOs: 13 and 10; c) SEQ ID NOs: 9 and 14; d) SEQ ID NOs: 9 and 15; or e) SEQ ID NOs: 9 and 16 wherein the CDR sequences are those indicated in claim 2; or iii) a conservatively substituted amino acid sequence of i) wherein the CDR sequences are those indicated in claim 2.

4. The antibody of claim 1, wherein
the VL domain comprises i) a polypeptide having an amino acid sequence of SEQ ID NO: 9; ii) a polypeptide having an amino acid sequence with at least 80%, at least 90%, or at least 95% sequence identity to SEQ ID NO: 9; or iii) a conservatively substituted amino acid sequence of SEQ ID NO: 9,
the VH domain comprises i) a polypeptide having an amino acid sequence of SEQ ID NO: 10; ii) a polypeptide having an amino acid sequence with at least 80%, at least 90%, or at least 95% sequence identity to SEQ ID NO: 10; or iii) a conservatively substituted amino acid sequence of SEQ ID NO: 10,
and wherein the antibody comprises one or more mutations at positions selected from R18, S50, and Y53 of SEQ ID NO: 9, and/or one or more mutations at positions selected from N30, Y34, D58, E61, E64, G101, and Q111 of SEQ ID NO: 10.

5. The anti-PF4 antibody of claim 4, wherein the one or more mutations are selected from R18K, S50N, and Y53H of SEQ ID NO: 9, and/or selected from N30K, Y34H, D58N, D58G, E61V, E64K, G101R, and Q111P of SEQ ID NO: 10.

6. The antibody of claim 5, wherein the one or more mutations are selected from the following combinations a), b), c), d), and e):
a) R18K of SEQ ID NO: 9 and N30K and D58N of SEQ ID NO: 10;
b) S50N and Y53H of SEQ ID NO: 9;
c) Y34H of SEQ ID NO: 10;
d) Y34H, D58G, and Q111P of SEQ ID NO: 10; and
e) E61V, E64K, and G101R of SEQ ID NO: 10.

7. The antibody of claim 1, wherein the antibody is an antibody fragment that does not comprise an Fc domain.

8. The antibody of claim 7, wherein the antibody is a scFv.

9. The antibody of claim 8, wherein the scFv comprises, from N-terminus to C-terminus, VL-linker-VH.

10. The antibody of claim 9, wherein the scFv comprises a polypeptide having an amino acid sequence of any one of SEQ ID NOs: 4-8.

11. A nucleic acid molecule encoding the antibody of claim 1.

12. The nucleic acid molecule of claim 11, having a sequence of any one of SEQ ID NOs: 33-37, or functional variants thereof.

13. A cell expressing the antibody of claim 1, or comprising a nucleic acid encoding said antibody.

14. A pharmaceutical composition comprising the antibody of claim 7 and a pharmaceutically acceptable carrier or excipient.

15. A method of diagnosing heparin-induced thrombocytopenia (HIT) in a patient, the method comprising:
a. obtaining a biological sample comprising patient antibodies to PF4/heparin from the patient;
b. contacting the sample with i) PF4/heparin in the presence of the isolated anti-PF4 antibody of claim 1, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, and ii) PF4/heparin in the absence of the isolated anti-PF4 antibody of claim 1, or the wildtype KKO antibody fragment, under conditions permissive for forming PF4/heparin:patient antibody complexes;
c. detecting the presence of any PF4/heparin:patient antibody complexes in i) and ii), wherein the detecting does not detect the isolated anti-PF4 antibody, or the wildtype KKO antibody fragment; and
d. determining the relative amount of PF4/heparin:patient antibody complexes in i) and ii) thereby determining if PF4/heparin:patient antibody binding is inhibited;
wherein the patient is diagnosed as having HIT if PF4/heparin:patient antibody binding is inhibited.

16. The method of claim 15, wherein the PF4/heparin is contacted with the isolated anti-PF4 antibody or the wildtype KKO antibody fragment prior to contacting with the sample.

17. The method of claim 15, wherein the biological sample comprises blood, serum, or plasma.

18. (canceled)

19. (canceled)

20. (canceled)

21. A method of treating or preventing heparin-induced thrombocytopenia, the method comprising administering a therapeutically effective amount of the antibody of claim 7 or a pharmaceutical composition comprising said antibody, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, to a subject in need thereof.

22. The method of claim 15, further comprising administering a therapeutically effective amount of the isolated anti-PF4 antibody wherein the isolated anti-PF4 antibody is an antibody fragment that does not comprise an Fc domain, or a wildtype KKO antibody fragment comprising a light chain variable (VL) domain comprising complementarity determining regions (CDRs) CDR-L1, CDR-L2, and CDR-L3 having amino acid sequences of SEQ ID NOs: 18-20, and a heavy chain variable (VH) domain comprising CDRs CDR-H1, CDR-H2, and CDR-H3 having amino acid sequences SEQ ID NOs: 21-23, to the patient.

23. A kit comprising i) the antibody of claim 1 or a binding fragment thereof, ii) a nucleic acid encoding said antibody or binding fragment thereof, iii) a composition comprising said antibody or binding fragment thereof, or iv) dosage form comprising said antibody or binding fragment thereof; and optionally a reference agent and/or instructions for use thereof.
